SEQUENCE LISTING (3 sequences)

SEQ ID NO: 1 - RNA, 1273 nt, Homo sapiens
  modified_base order(19,25,38,43,44,46,52,58,64,66,71,78,84,96,99,101,105,108,111,114,125,126,129,141,150,153,159,171,174,183,204,213,222,225,237,246,251,252,255,267,270,275,284,297,300,306,308,324,327,330,362,363,371,381,383,395,396,411,413,420,422,426,435,453,455,456,458,465,471,485,492,495,519,524,525,536,545,557,558,578,581,585,596,597,600,602,603,609,626,630,633,639,645,648,653,671,680,689,690,696,716,723,725,729,768,771,774,788,789,818,822,824,830,837,840,843,854,857,860,861,869,885,894,896,897,899,927,936,941,947,951,965,972,987,990,993,1002,1007,1008,1010,1016,1019,1025,1029,1033,1037,1040,1043,1047,1051,1052,1057,1060,1074,1077,1083,1086,1088,1092,1094,1095,1097,1100,1102,1103,1105,1110,1111,1113,1124,1131,1136,1138,1139,1141,1143,1144): mod_base = OTHER; note = 5-methoxy-pseudouridine
  source 1..1273: mol_type = genomic RNA
ggggaggaaccgagaggctgagactaacccagaaacatccaattctcaaactgaagctcgcactctcgcctccagcatgaaggtgagcgccgccctgctgtgcctgctgctgatcgccgccaccttcatcccccagggcctggccggcatcctggacctgggcccccagatgctgcgcgagctgcaggagaccaacgccgccctgcaggacgtgcgcgagctgctgcgccagcaggtgcgcgagatcaccttcctgaagaacaccgtgatggagtgcgacgcctgcggcggcagcctggtgaacctgtgcaccaaggagggcgtgctgctgaagggcggcaagcgcgaggaggagaagcccttccgcgactgcgccgacgtgtaccaggccggcttcaacaagagcggcatctacaccatctacatcaacaacatgcccgagcccaagaaggtgttctgcaacatggacgtgaacggcggcggctggaccgtgatccagcaccgcgaggacggcagcctggacttccagcgcggctggaaggagtacaagatgggcttcggcaaccccagcggcgagtactggctgggcaacgagttcatcttcgccatcaccagccagcgccagtacatgctgcgcatcgagctgatggactgggagggcaaccgcgcctacagccagtacgaccgcttccacatcggcaacgagaagcagaactaccgcctgtacctgaagggccacaccggcaccgccggcaagcagagcagcctgatcctgcacggcgccgacttcagcaccaaggacgccgacaacgacaactgcatgtgcaagtgcgccctgatgctgaccggcggctggtggttcgacgcctgcggccccagcaacctgaacggcatgttctacaccgccggccagaaccacggcaagctgaacggcatcaagtggcactacttcaagggccccagctacagcctgcgcagcaccaccatgatgatccgccccctggacttctaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 907 nt, Homo sapiens
  source 1..907: mol_type = genomic RNA
  modified_base order(19,25,38,43,44,46,52,58,64,66,71,78,84,86,88,96,97,99,101,105,108,111,114,125,126,129,130,141,145,159,162,164,177,180,188,192,195,219,230,242,249,261,267,282,287,288,290,296,306,312,327,333,335,345,351,354,366,369,384,387,390,407,417,423,429,441,450,459,468,471,480,501,534,543,557,558,567,569,570,587,588,591,603,609,611,626,644,650,653,659,663,667,671,674,677,681,685,686,691,694,708,711,717,720,722,726,728,729,731,734,736,737,739,744,745,747,758,765,770,772,773,775,777,778): mod_base = OTHER; note = 5-methoxy-pseudouridine
ggggaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgcccccccccggctgatctgcgacagccgggtgctggagcggtacctgctggaggccaaggaggccgagaacatcaccaccggctgcgccgagcactgcagcctgaacgagaacatcaccgtgcccgacaccaaggtgaacttctacgcctggaagcggatggaggtgggccagcaggccgtggaggtgtggcagggcctggccctgctgagcgaggccgtgctgcggggccaggccctgctggtgaacagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccggaccggcgaccggtgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - RNA, 720 nt, Homo sapiens
  modified_base order(2,5,23,25,26,35,38,44,47,50,56,68,82,83,89,91,111,118,128,134,139,140,143,145,161,167,172,182,185,194,199,206,211,214,215,223,236,250,251,253,254,259,266,277,281,296,298,299,301,302,319,338,343,344,359,362,371,377,386,391,392,401,413,425,430,436,452,454,456,458,461,485,491,496,497,503,515,530,536,547,566,581,584,587,601,605,616,623,651,656,659,662,665,670,671,674,689,693,695,701,710,712,718): mod_base = OTHER; note = 5-methoxy-pseudouridine
  source 1..720: mol_type = genomic RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

(19) United States
(12) Patent Application Publication
(10) Pub. No.: US 2023/0263819 A1
(43) Pub. Date: Aug. 24, 2023

(54) Lipid Composition and Use Thereof for Delivery of a Therapeutically Active Agent to Endothelium

(71) Applicant: Pantherna Therapeutics GmbH, Hennigsdorf (DE)

(72) Inventors: Volker FEHRING, Berlin (DE); Jörg KAUFMANN, Berlin (DE); Oliver KEIL, Berlin (DE); Daniel TONDERA, Berlin (DE)

(21) Appl. No.: 17/884,997

(22) Filed: Aug. 10, 2022

Related U.S. Application Data
(63) Continuation of application No. PCT/EP2021/053387, filed on Feb. 11, 2021.

(30) Foreign Application Priority Data
Feb. 11, 2020 (EP) 20000065.1

(51) Int. Cl. A61K 31/7105; A61K 47/69; A61K 47/12; A61K 9/127

(52) U.S. Cl. CPC A61K 31/7105; A61K 47/6911; A61K 47/12; A61K 9/1272

(57) ABSTRACT

The present invention is related to a composition comprising a lipid composition, a tricarboxylic acid and a nucleic acid molecule, wherein the lipid composition comprises a cationic lipid, a neutral lipid and a shielding lipid, wherein a positive charge excess arising from a larger number of positive charges provided by the cationic lipid molecules in the composition compared to the smaller number of negative charges provided by the nucleic acid molecules in the composition is compensated by the charges provided by the tricarboxylic acid; and methods of use of such composition.

### RELATED APPLICATIONS

This application is a continuation of PCT/EP2021/053387, filed Feb. 11, 2021, which claims priority to EP20000065.1, filed Feb. 11, 2020, the contents of each of which are hereby incorporated by reference in their entireties.

### SEQUENCE LISTING

The instant application contains a sequence listing, submitted electronically in ST.26 (XML) format, which is hereby incorporated by reference in its entirety. The ST.26 sequence listing, created on May 8, 2023, is named 3956.0009C_ST.26_Sequence_List_MAY_8.XML.1 and is 10 kilobytes in size.

The present invention is related to a composition comprising a lipid composition; the composition comprising a lipid composition for use in a method for the treatment of a disease; use of the composition comprising a lipid composition for the manufacture of a medicament for the treatment and/or prevention of a disease; a pharmaceutical composition comprising a composition comprising a lipid composition; a kit comprising the composition comprising a lipid composition; a method for the treatment and/or prevention of a disease, wherein the method comprises administering to a subject in need thereof an effective amount of the composition comprising a lipid composition, and a method for preparing the composition

Both molecular biology as well as molecular medicine heavily rely on the introduction of biologically active compounds into cells. Such biologically active compounds typically comprise, among others, DNA, RNA as well as peptides and proteins, respectively. The barrier which has to be overcome is typically a lipid bilayer which has a negatively charged outer surface. In the art, a number of technologies have been developed to penetrate the cellular membrane and to thus introduce the biologically active compounds. Some methods conceived for laboratory use, however, cannot be used in the medical field and are more particularly not suitable for drug delivery. For example, electroporation and ballistic methods known in the art, would, if at all, only allow a local delivery of biologically active compounds. Apart from said lipid bilayer cellular membranes also comprise transporter systems. Accordingly, efforts were undertaken to use this kind of transporter systems in order to transfer the biologically active compounds across the cell membrane. However, due to the specificity or cross-reactivity of such transporter systems, their use is not a generally applicable method.

A more generally applicable approach described in the art for transferring biologically active compounds into cells, is the use of viral vectors. However, viral vectors can be used only for transferring genes efficiently into some cell types; but they cannot be used to introduce chemically synthesized molecules into the cells.

An alternative approach was the use of so-called liposomes (Bangham, J. Mol. Biol. 13, 238-252). Liposomes are vesicles which are generated upon association of amphiphilic lipids in water. Liposomes typically comprise concentrically arranged bilayers of phospholipids. Depending on the number of layers liposomes can be categorized as small unilamelar vesicles, multilamelar vesicles and large multilamelar vesicles. Liposomes have proven to be effective delivery agents as they allow incorporating hydrophilic compounds into the aqueous intermediate layers, whereas hydrophobic compounds are incorporated into the lipid layers. It is well known in the art that both the composition of the lipid formulation as well as its method of preparation have an effect on the structure and size of the resultant lipid aggregates and thus on the liposomes. Liposomes are also known to incorporate cationic lipids.

Cationic lipids have, apart from being components of liposomes, also attracted considerable attention as they may as such be used for cellular delivery of biopolymers. Using cationic lipids any anionic compound can be encapsulated essentially in a quantitative manner due to electrostatic interaction. In addition, it is believed that the cationic lipids interact with the negatively charged cell membranes initiating cellular membrane transport. It has been found that the use of a liposomal formulation containing cationic lipids or the use of cationic lipids as such together with a biologically active compound requires a heuristic approach as each formulation is of limited use because it typically can deliver plasmids into some but not all cell types, usually in the absence of serum.

Charge and/or mass ratios of lipids and the biologically active compounds to be transported by them have turned out to be a crucial factor in the delivery of different types of said biologically active compounds. For example, it has been shown that lipid formulations suitable for plasmid delivery comprising 5,000 to 10,000 bases in size, are generally not effective for the delivery of oligonucleotides such as siRNA molecules, synthetic ribozymes or antisense molecules typically comprising about 10 to about 50 bases. In addition, it has recently been indicated that optimal delivery conditions for antisense oligonucleotides and ribozymes are different, even in the same cell type.

U.S. Pat. No. 6,395,713 discloses cationic lipid-based compositions whereby the cationic lipid consists of a lipophilic group, a linker and a head group and the use of such compositions for transferring biologically active compounds into a cell.

International patent application WO 2005/105152 discloses another cationic lipid-based composition which proved to be particularly effective in the delivery of functional nucleic acid molecule such as siRNA molecules.

Depending on the disease to be treated and the drug to be delivered, there is a need for delivering the drug to specific organs or specific cell types. One such specific organ is lung and one such specific cell type is pulmonary endothelial cell. The targeting of lung and pulmonary endothelial cell is, for example, advantageous in the delivery of a drug for the treatment of a disease such as acute lung injury, acute respiratory distress syndrome, lung cancer, pulmonary metastasis, pulmonary hypertension and pulmonary artery hypertension.

A problem underlying the present invention is the provision of a means capable of delivering an agent, preferably a therapeutically active agent, more preferably a drug, to lung. A further problem underlying the present invention is the provision of a means capable of delivering an agent, preferably a therapeutically active agent, more preferably a drug, to lung tissue. A still further problem underlying the present invention is the provision of a means which is capable of delivering an agent, preferably a therapeutically active agent, more preferably a drug, to a pulmonary endothelial cell. In connection with each and any of these problems, the agent and drug, respectively, is preferably an mRNA.

Another problem underlying the present invention is the provision of a means for the treatment of a lung disease, preferably a lung disease which is selected from the group comprising acute lung injury, acute respiratory distress syndrome, lung cancer, pulmonary metastasis, pulmonary hypertension and pulmonary artery hypertension. In connection with this problem, the agent and drug, respectively, is preferably an mRNA.

Another problem underlying the present invention is the provision of a delivery vehicle as part of a means for the treatment of a lung disease, preferably a lung disease which is selected from the group comprising acute lung injury, acute respiratory distress syndrome, lung cancer, pulmonary metastasis, pulmonary hypertension and pulmonary artery hypertension. In connection with this problem, the agent and drug, respectively, is preferably an mRNA.

Another problem underlying the present invention is the provision of a pharmaceutical composition. Preferable, the pharmaceutical is suitable for the delivery of an agent, preferably a therapeutically active agent, more preferably a drug, to lung. A further problem underlying the present invention is the provision of a pharmaceutical composition suitable for the delivery of an agent, preferably a therapeutically active agent, more preferably a drug, to lung tissue. A still further problem underlying the present invention is the provision of a pharmaceutical composition suitable for the delivery of an agent, preferably a therapeutically active agent, more preferably a drug, to a pulmonary endothelial cell. In connection with each and any of these problems, the agent and drug, respectively, is preferably an mRNA.

Another problem underlying the present invention is the provision of a means which can be used in the manufacture of a medicament, wherein the medicament is suitable for or is for use in the treatment of lung disease, preferably a lung disease which is selected from the group comprising acute lung injury, acute respiratory distress syndrome, lung cancer, pulmonary metastasis, pulmonary hypertension and pulmonary artery hypertension. In connection with this problem, the agent and drug, respectively, is preferably an mRNA.

Another problem underlying the present invention is the provision of a method and/or prevention for the treatment of a disease, wherein the method comprises administering to a subject in need thereof an effective amount a composition comprising a therapeutically or pharmaceutically active agent, preferably a drug. A further problem underlying the present invention is the provision of a method for the treatment and/or prevention of a lung disease, wherein the method comprises administering to a subject in need thereof an effective amount a composition comprising a therapeutically or pharmaceutically active agent, preferably a drug. A still further problem underlying the present invention is the provision of a method for the treatment and/or prevention of a disease, preferably a lung disease, whereby the treatment comprises delivering a therapeutically or pharmaceutically active agent to the lung, preferably to a pulmonary endothelial cell. In connection with each and any of these problems, the agent and drug, respectively, is preferably an mRNA.

Another problem underlying the present invention is the provision of a kit. Preferably, the kit is suitable (a) for use in a method for the treatment and/or prevention of a disease, preferably a lung disease, (b) for use in a method of transferring a biologically active agent, a therapeutically active agent and/or or pharmaceutically active agent into a cell or across a membrane of a cell, whereby preferably such cell is a pulmonary endothelial cell, and/or (c) for use in the manufacture of a medicament, preferably a medicament for the treatment and/or prevention of a disease, more preferably a lung disease and most preferably a lung disease selected from the group comprising acute lung injury, acute respiratory distress syndrome, lung cancer, pulmonary metastasis, pulmonary hypertension and pulmonary artery hypertension. In connection with each and any of these problems, the agent and drug, respectively, is preferably an mRNA.

Another problem underlying the present invention is the provision of a means for the effective transfection of a plurality of cells, preferably pulmonary endothelial cells, whereby starting from a given amount of lipid composition and a given amount of a nucleic acid such as an mRNA, said amount of nucleic acid is transfected into a bigger part of the plurality of cells. Preferably, the means is a means as subject to the other problems underlying the present invention.

A still further problem underlying the present invention is the provision of a means for increasing the number of lipid nanoparticles which can be produced at a given amount, preferably a given molar amount of a nucleic acid such as an mRNA, whereby such lipid nanoparticles are capable of efficiently transfecting cells and/or introducing an effective amount of the nucleic acid into cells, preferably pulmonary endothelial cells.

Another problem underlying the present invention is the provision of a means for generating lipid nanoparticles having a monodisperse size distribution.

A still further problem underlying the present invention is the provision of a means for increasing the number of lipid nanoparticles which can be produced at a given amount, preferably a molar amount of a nucleic acid such as an mRNA, whereby such lipid nanoparticles are capable of efficiently transfecting cells and/or introducing an effective amount of the nucleic acid into cells, preferably pulmonary endothelial cells, and whereby the lipid nanoparticles have a monodisperse size distribution.

A further problem underlying the present invention is the provision of a means which allows increased release of a nucleic acid molecule such as an mRNA from the endosome of a cell. Preferably, such means is a lipid formulation, more preferably a lipid nanoparticle comprising a nucleic acid, preferably an mRNA, the release of which from the endosome is increased upon transfection of a cell in vitro or in vivo.

These and other problems are solved by the subject matter of the attached independent claims. Preferred embodiments may be taken from the attached dependent claims. These and other problems are also solved by the following embodiments.

Embodiment 1. A composition comprising a lipid composition, a tricarboxylic acid and a nucleic acid molecule, wherein the lipid composition comprises a cationic lipid, a neutral lipid and a shielding lipid.

Embodiment 2. The composition of Embodiment 1, wherein a positive charge excess arising from a larger number of positive charges provided by the cationic lipid molecules in the composition compared to the smaller number of negative charges provided by the nucleic acid molecules in the composition is compensated by the charges provided by the tricarboxylic acid.

Embodiment 3. The composition of any one of Embodiments 1 to 2, wherein the nucleic acid molecule is an mRNA molecule.

Embodiment 4. The composition of any one of Embodiments 1, 2 and 3, wherein the composition is a monodisperse composition.

Embodiment 5. The composition of Embodiment 4, wherein the amount of the tricarboxylic acid in the composition is such that the composition is a monodisperse composition.

Embodiment 6. The composition of any one of Embodiments 4 and 5, wherein the amount of the tricarboxylic acid in the composition is higher than the concentration of the tricarboxylic acid at which the composition is a polydisperse composition.

Embodiment 7. The composition of any one of Embodiments 1 to 6, wherein the ratio of the mass of total lipids in the formulation to mass of a nucleic acid molecule, preferably an mRNA molecule, in the composition (m/m ratio (total lipids/mRNA)) is from 10 to 140.

Embodiment 8. The composition of Embodiment 7, wherein the m/m ratio (total lipids/mRNA) is from 10 to 20, preferably 12 to 16, and more preferably 14.

Embodiment 9. The composition of Embodiment 8, wherein the composition comprises an amount of a nucleic acid molecule, preferably an amount of an mRNA molecule, wherein the amount of the nucleic acid molecule, preferably of the mRNA molecule, of the composition is from about 0.01 mg/ml to about 1.5 mg/ml, preferably from about 0.05 mg/ml to about 0.8 mg/ml, more preferably from about 0.075 mg/ml to about 0.4 mg/ml, and most preferably about 0.2 mg/ml.

Embodiment 10. The composition of Embodiment 7, wherein the m/m ratio (total lipids/mRNA) is from 20 to 40, preferably from 24 to 32, and more preferably 28.

Embodiment 11. The composition of Embodiment 10, wherein the composition comprises an amount of a nucleic acid molecule, preferably an amount of an mRNA molecule, wherein the amount of the nucleic acid molecule, preferably of the mRNA molecule, of the composition is from about 0.01 mg/ml to about 1.5 mg/ml, preferably from about 0.05 mg/ml to about 0.8 mg/ml, more preferably from about 0.075 mg/ml to about 0.4 mg/ml, and most preferably about 0.2 mg/ml.

Embodiment 12. The composition of Embodiment 7, wherein the m/m ratio (total lipids/mRNA) is from 40 to 80, preferably from 48 to 64, more preferably 56.

Embodiment 13. The composition of Embodiment 12, wherein the composition comprises an amount of a nucleic acid molecule, preferably an amount of an mRNA molecule, wherein the amount of the nucleic acid molecule, preferably of the mRNA molecule, of the composition is from about 0.01 mg/ml to about 1 mg/ml, preferably from about 0.05 mg/ml to about 0.8 mg/ml, more preferably from about 0.075 mg/ml to about 0.4 mg/ml, and most preferably about 0.15 mg/ml.

Embodiment 14. The composition of Embodiment 7, wherein the m/m ratio (total lipids/mRNA) is from 80 to 140, preferably from 96 to 128, more preferably 112.

Embodiment 15. The composition of Embodiment 14, wherein the composition comprises an amount of a nucleic acid molecule, preferably an amount of an mRNA molecule, wherein the amount of the nucleic acid molecule, preferably of the mRNA molecule, of the composition is from about 0.01 mg/ml to about 0.65 mg/ml, preferably from about 0.05 mg/ml to about 0.5 mg/ml, more preferably from about 0.075 mg/ml to about 0.3 mg/ml, and most preferably about 0.1 mg/ml.

Embodiment 16. The composition of any one of Embodiments 7 to 15, preferably any one of Embodiments 8 and 9, wherein the concentration of the tricarboxylic acid in the formulation is from 1.35 μmol/ml to 3.23 μmol/ml, preferably from 1.72 μmol/ml to 2.86 μmol/ml.

Embodiment 17. The composition of any one of Embodiments 7 to 15, preferably any one of Embodiments 10 and 11, wherein the concentration of the tricarboxylic acid in the formulation is from 2.76 μmol/ml to 6.40 μmol/ml, preferably from 3.55 μmol/ml to 5.73 μmol/ml.

Embodiment 18. The composition of any one of Embodiments 7 to 15, preferably any one of Embodiments 12 and 13, wherein the concentration of the tricarboxylic acid in the formulation is from 5.52 μmol/ml to 12.80 μmol/ml, preferably from 6.87 μmol/ml to 11.45 μmol/ml.

Embodiment 19. The composition of any one of Embodiments 7 to 15, preferably any one of Embodiments 14 and 15, wherein the concentration of the tricarboxylic acid in the formulation is from 10.98 μmol/ml to 25.66 μmol/ml, preferably from 13.64 μmol/ml to 22.90 μmol/ml.

Embodiment 20. The composition of any one of Embodiments 1 to 15, preferably any one of Embodiments 7 to 19, more preferably of any one of Embodiments 16 to 19, wherein the composition comprise an amount of a nucleic acid molecule, preferably mRNA molecule, wherein the amount of mRNA molecule of the composition is from about 0.01 mg/ml to about 1.5 mg/ml, preferably from 0.05 mg/ml to about 0.8 mg/ml, more preferably from about 0.075 mg/ml to about 0.4 mg/ml, and most preferably about 0.2 mg/ml.

Embodiment 21. The composition of any one of Embodiments 1 to 20, preferably any one of Embodiments 7 to 20, more preferably of any one of Embodiments 16 to 20, wherein the lipid composition forms particles.

Embodiment 22. The composition of Embodiment 21, wherein the particles comprise the tricarboxylic acid.

Embodiment 23. The composition of Embodiment 22, wherein the tricarboxylic acid is contained in the particles.

Embodiment 24. The composition of any one of Embodiments 1 to 23, wherein the tricarboxylic acid forms a complex with the lipid composition.

Embodiment 25. The composition of any one of Embodiments 22 to 24, wherein the tricarboxylic acid is bound in or bound to the particles.

Embodiment 26. The composition of Embodiment 25, wherein the tricarboxylic acid is bound by or bound to the particles after dialysis of the composition and/or the particles.

Embodiment 27. The composition of Embodiment 21, wherein the particles comprise the nucleic acid molecule, preferably the mRNA molecule.

Embodiment 28. The composition of any one of Embodiments 21 to 27, wherein the nucleic acid molecule, preferably the mRNA molecule, forms part of the particles.

Embodiment 29. The composition of any one of Embodiments 1 to 28, wherein the nucleic acid molecule, preferably the mRNA molecule, is therapeutically active.

Embodiment 30. The composition of any one of Embodiments 1 to 29, wherein the particle size is from 30 nm to 200 nm, preferably from about 40 nm to about 140 nm, and even more preferable from about 60 nm to 120 nm.

Embodiment 31. The composition of Embodiment 30, wherein the particle size is determined by dynamic light scattering.

Embodiment 32. The composition of any one of Embodiments 21 to 31, wherein the particles have a zeta potential, wherein the zeta potential of the particles is about +0 mV to about +80 mV, preferably the zeta potential of the particles is about +0 mV to about +45 mV.

Embodiment 33. The composition of Embodiment 32, wherein the zeta potential is measured by Laser Doppler Electrophoresis measurement.

Embodiment 34. The composition of any one of Embodiments 1 to 33, preferably any one of Embodiments 7 to 20, wherein the tricarboxylic acid is selected from the group comprising citric acid, isocitric acid (1-hydroxypropane-1,2,3-tricarboxylic acid), cis-aconitic acid, trans-aconitic acid, a mixture of both cis-aconitic acid and trans-aconitic acid, propane-1,2,3-tricarboxylic acid (tricarballylic acid), agaric acid, trimesic acid, and any mixture thereof.

Embodiment 35. The composition of 34, wherein the tricarboxylic acid is citric acid.

Embodiment 36. The composition of any one of Embodiments 1 to 35, wherein the composition comprises a carrier, preferably a pharmaceutically acceptable carrier.

Embodiment 37. The composition of Embodiment 36, wherein the carrier is selected from the group comprising water, an aqueous solution, preferably an isotonic aqueous solution, a sucrose solution, preferably an isotonic sucrose solution, a salt solution, preferably an isotonic salt solution, a buffer solution, preferably an isotonic buffer solution and a water miscible solvent.

Embodiment 38. The composition of Embodiment 37, wherein the carrier is selected from the group comprising an isotonic aqueous solution, an isotonic aqueous solution, an isotonic sucrose solution, an isotonic salt solution and an isotonic buffer solution.

Embodiment 39. The composition of any one of Embodiments 36 to 38, wherein the carrier is an aqueous sucrose solution, preferably a 270 mM aqueous sucrose solution or a 270 mM sucrose solution in a buffer, preferably the buffer is a 10 mM TRIS buffer pH 7.4.

Embodiment 40. The composition of any one of Embodiments 1 to 39, wherein cationic lipid is selected from the group comprising β-(L-Arginyl)-L-2,3-diamino propionic acid-N-palmityl-N-oleyl-amide, DOTAP (N-[1-(2,3-Dioleoyloxy)propyl]-N,N,N-trimethylammonium methyl-chloride), DOTMA (1,2-Di-O-octadecenyl-3-trimethylammonium propane (preferably as chloride salt)) and DC-cholesterol (3β[N-(N′,N′-Dimethylaminoethane)-carbamoyl]cholesterol), preferably the cationic lipid is β-(L-Arginyl)-L-2,3-diamino propionic acid-N-palmityl-N-oleyl-amide or

Embodiment 41. The composition of any one of Embodiments 1 to 40, preferably Embodiment 40, wherein the neutral lipid is (a) a zwitterionic phospholipid selected from the group comprising Diphytanoyl-PE (1,2-Diphytanoyl-sn-glycero-3-phosphoethanolamine), DOPE (1,2-Dioleoyl-sn-glycero-3-phosphoethanolamine), DLPE (1,2-Dilauroyl-sn-glycero-3-phosphoethanolamine), DMPE ((1,2-Dimyristoyl-sn-glycero-3-phosphoethanolamine)), POPE ((1-Palmitoyl-2-oleoyl-sn-glycero-3-phosphoethanolamine)) and DSPE (1,2-Distearoyl-sn-glycero-3-phosphoethanolamine), or

(b) an uncharged sterol lipid selected from a group comprising cholesterol and stigmasterol, preferably, the phospholipid Diphytanoyl-PE.

Embodiment 42. The composition of any one of Embodiments 1 to 41, preferably Embodiment 41, wherein the shielding lipid is a PEGylated lipid selected from the group comprising methoxyPEG-DSPE ((1,2-Distearoyl-sn-glycero-3-phosphoethanolamine-N-[methoxy(polyethylene glycol)] (preferably as ammonium or sodium salt), methoxyPEG-DLG (1,2 -Dilauroyl-rac-glycero-3-methoxypolyethylene glycol), methoxyPEG-DMG (1,2 -Dimyristoyl-rac-glycero-3 -methoxypolyethylene glycol), methoxyPEG-DPG (1,2 -Dipalmitoyl-rac-glycero-3-methoxypolyethylene glycol), methoxyPEG-DSG (1,2 -Distearoyl-rac-glycero-3 -methoxypolyethylene glycol), methoxyPEG-c-DMA (N-[(Methoxy poly(ethylene glycol))carbamyl]-1,2-dimyristyloxlpropyl-3-amine), methoxy PEG-C8-ceramide (N-Octanoyl-sphingosine-1-{succinyl[methoxy (polyethylene glycol)]}) and methoxyPEG-C16-ceramide (N-Palmitoyl-sphingosine-1-{succinyl[methoxy (polyethylene glycol)]}).

Embodiment 43. The composition of any one of Embodiments 1 to 42, preferably any one of Embodiments 40 to 42, more preferably Embodiment 42, wherein the shielding lipid is a PEGylated lipid, preferably the PEGylated lipid is methoxyPEG2000-DSPE.

Embodiment 44. The composition of any one of Embodiments 1 to 43, wherein the molar ratio of the lipids in the lipid composition is
from about 20 mol-% to about 80 mol-% of the cationic lipid,
from about 10 mol-% to about 70 mol-% of the neutral lipid, and
from about 0.1 mol-% to 10 mol-%, preferably from about 1 mol-% to about 10 mol-% of the shielding lipid, wherein preferably shielding lipid is a PEGylated lipid, wherein the overall lipid content is 100%.

Embodiment 45. The composition of Embodiment 44, wherein the molecular weight of the PEG of the PEGylated lipid is from about 750 Da to about 5000 Da, preferably from about 1500 Da to 3000 Da.

Embodiment 46. The composition of Embodiment 45, wherein the molar ratio of the lipids in the lipid composition is
from about 35 mol-% to about 65 mol-% of the cationic lipid,
from about 35 mol-% to about 65 mol-% of the neutral lipid, and
from about 0.1 mol-% to 5 mol-% of the shielding lipid, preferably of the PEGylated lipid,
   wherein the overall lipid content is 100%,
   preferably the molar ratio of the lipids in the composition is
from about 45 mol-% to about 55 mol-% of the cationic lipid,
from about 45 mol-% to about 55 mol-% of the neutral lipid, and
from about 0.5 mol-% to 2 mol-%,
   wherein the overall lipid content is 100%.

Embodiment 47. The composition of any one of Embodiments 1 to 46, preferably any one of Embodiments 44 to 46, wherein the molar ratio of the lipid composition is
50 mol-% of the cationic lipid, wherein the cationic lipid is β-(L-Arginyl)-L-2,3-diamino propionic acid-N-palmityl-N-oleyl-amide or L-Arginyl-P-alanine-N-palmityl-N-oleyl-amide,
49 mol-% of the neutral lipid, wherein the neutral lipid is Diphytanoyl-PE (1,2-Diphytanoyl-sn-glycero-3-phosphoethanolamine), and
1 mol-% of the shielding lipid, wherein the shielding lipid is mPEG-2000-DSPE (1,2-distearoyl-sn-glycero-3-phosphoethanolamine-N-[methoxy(polyethylene glycol)-2000] (preferably as sodium salt)).

Embodiment 48. The composition of Embodiment 47, wherein the ratio of the mass of total lipids in the formulation to mass of nucleic acid molecule, preferably mRNA molecule, in the composition (m/m ratio (total lipids/mRNA)) is from 10 to 140.

Embodiment 49. The composition of Embodiment 48, wherein the m/m ratio (total lipids/mRNA) is from 10 to 20, preferably 12 to 16, and more preferably 14.

Embodiment 50. The composition of Embodiment 49, wherein the composition comprises an amount of a nucleic acid molecule, preferably an amount of an mRNA molecule, wherein the amount of the nucleic acid molecule, preferably of the mRNA molecule, of the composition is from about 0.01 mg/ml to about 1.5 mg/ml, preferably from about 0.05 mg/ml to about 0.8 mg/ml, more preferably from about 0.075 mg/ml to about 0.4 mg/ml; and most preferably about 0.2 mg/ml.

Embodiment 51. The composition of Embodiment 48, wherein the m/m ratio (total lipids/mRNA) is from 20 to 40, preferably from 24 to 32, and more preferably 28.

Embodiment 52. The composition of Embodiment 51, wherein the composition comprises an amount of a nucleic acid molecule, preferably an amount of an mRNA molecule, wherein the amount of the nucleic acid molecule, preferably of the mRNA molecule, of the composition is from about 0.01 mg/ml to about 1.5 mg/ml, preferably from about 0.05 mg/ml to about 0.8 mg/ml, more preferably from about 0.075 mg/ml to about 0.4 mg/ml; and most preferably about 0.2 mg/ml.

Embodiment 53. The composition of Embodiment 48, wherein the m/m ratio (total lipids/mRNA) is from 40 to 80, preferably from 48 to 64, more preferably 56.

Embodiment 54. The composition of Embodiment 53, wherein the composition comprises an amount of a nucleic acid molecule, preferably an amount of an mRNA molecule, wherein the amount of the nucleic acid molecule, preferably of the mRNA molecule, of the composition is from about 0.01 mg/ml to about 1 mg/ml, preferably from about 0.05 mg/ml to about 0.8 mg/ml, more preferably from about 0.075 mg/ml to about 0.4 mg/ml; and most preferably about 0.15 mg/ml.

Embodiment 55. The composition of Embodiment 48, wherein the m/m ratio (total lipids/mRNA) is from 80 to 140, preferably from 96 to 128, more preferably 112.

Embodiment 56. The composition of Embodiment 55, wherein the composition comprises an amount of a nucleic acid molecule, preferably an amount of an mRNA molecule, wherein the amount of the nucleic acid molecule, preferably of the mRNA molecule, of the composition is from about 0.01 mg/ml to about 0.65 mg/ml, preferably from about 0.05 mg/ml to about 0.5 mg/ml, more preferably from about 0.075 mg/ml to about 0.3 mg/ml; and most preferably about 0.1 mg/ml.

Embodiment 57. The composition of any one of Embodiments 48 to 56, preferably any one of Embodiments 49 and 50, wherein the concentration of the tricarboxylic acid in the formulation is from 1.35 μmol/ml to 3.23 μmol/ml, preferably from 1.72 μmol/ml to 2.86 μmol/ml.

Embodiment 58. The composition of any one of Embodiments 48 to 56, preferably any one of Embodiments 51 and 52, wherein the concentration of the tricarboxylic acid in the formulation is from 2.76 μmol/ml to 6.40 μmol/ml, preferably from 3.55 μmol/ml to 5.73 μmol/ml.

Embodiment 59. The composition of any one of Embodiment 48 to 56, preferably any one of Embodiments 53 and 54, wherein the concentration of the tricarboxylic acid in the formulation is from 5.52 μmol/ml to 12.80 μmol/ml, preferably from 6.87 μmol/ml to 11.45 μmol/ml.

Embodiment 60. The composition of any one of Embodiments 48 to 56, preferably any one of Embodiments 55 and 56, wherein the concentration of the tricarboxylic acid in the formulation is from 10.98 μmol/ml to 25.66 μmol/ml, preferably from 13.64 μmol/ml to 22.90 μmol/ml.

Embodiment 61. The composition of any one of Embodiments 44 to 60, preferably of any one of Embodiments 47 to 60, more preferably any one of Embodiments 57 to 60, wherein the composition comprises an amount of a nucleic acid molecule, preferably of an mRNA molecule, wherein the amount of the nucleic acid molecule, preferably of the mRNA molecule of the composition is from about 0.01 mg/ml to about 1.5 mg/ml, preferably from about 0.05 mg/ml to about 0.8 mg/ml, more preferably from about 0.075 mg/ml to about 0.4 mg/ml, and most preferably about 0.2 mg/ml.

Embodiment 62. The composition of any one of Embodiments 1 to 61, wherein the composition is suitable for delivering a therapeutically active agent to an endothelial cell.

Embodiment 63. The composition of Embodiment 62, wherein the endothelial cell is a pulmonary endothelial cell.

Embodiment 64. The composition of any one of Embodiments 1 to 63, wherein the composition is suitable for delivering a therapeutically active agent to vasculature, preferably pulmonary vasculature, more preferably to human pulmonary vasculature.

Embodiment 65. A composition comprising a lipid composition, wherein the lipid composition comprises a cationic lipid, a neutral lipid and a shielding lipid, wherein the cationic lipid is selected from the group comprising β-(L-Arginyl)-L-2,3-diamino propionic acid-N-palmityl-N-oleyl-amide, L-Arginyl-β-alanine-N-palmityl-N-oleyl-amide, DOTAP (N-[1-(2,3-Dioleoyloxy)propyl]-N,N,N-trimethylammonium methyl-chloride), DOTMA (1,2-Di-O-octadecenyl-3-trimethylammonium propane (preferably as chloride salt)) and DC-cholesterol (3β[N-(N′,N′-Dimethylaminoethane)-carbamoyl]cholesterol),

wherein the neutral lipid is a zwitterionic phospholipid selected from the group comprising Diphytanoyl-PE (1,2-Diphytanoyl-sn-glycero-3-phosphoethanolamine), DOPE (1,2-Dioleoyl-sn-glycero-3-phosphoethanolamine), DLPE (1,2-Dilauroyl-sn-glycero-3-phosphoethanolamine), DMPE ((1,2-Dimyristoyl-sn-glycero-3-phosphoethanolamine)), POPE ((1-Palmitoyl-2-oleoyl-sn-glycero-3-phosphoethanolamine)) and DSPE (1,2-Distearoyl-sn-glycero-3-phospho), or
an uncharged sterol lipid selected from a group comprising cholesterol and stigmasterol,
wherein the shielding lipid is a PEGylated lipid selected from the group comprising methoxyPEG-DSPE ((1,2-Distearoyl-sn-glycero-3-phosphoethanol amine-N-[methoxy(polyethylene glycol)] (preferably as ammonium or sodium salt), methoxyPEG-DLG (1,2-Dilauroyl-rac-glycero-3-methoxypolyethylene glycol), methoxyPEG-DMG (1,2-Dimyristoyl-rac-glycero-3-methoxypolyethylene glycol), methoxyPEG-DPG (1,2-Dipalmitoyl-rac-glycero-3-methoxypolyethylene glycol), methoxyPEG-DSG (1,2-Distearoyl-rac-glycero-3-methoxypolyethylene glycol), methoxyPEG-c-DMA (N-[(Methoxy poly(ethylene glycol))carbamyl]-1,2-dimyristyloxlpropyl-3-amine), methoxyPEG-C8-ceramide (N-Octanoyl-sphingosine-1-{succinyl[methoxy (polyethyleneglycol)]}), methoxyPEG-C16-ceramide (N-Palmitoyl-sphingosine-1-{succinyl[methoxy (polyethylene glycol)]}), and
wherein the composition comprises an mRNA.

Embodiment 66. The composition comprising a lipid composition, wherein the lipid composition comprises a cationic lipid, a neutral lipid and a shielding lipid, wherein the cationic lipid is selected from the group comprising β-(L-Arginyl)-L-2,3-diamino propionic acid-N-palmityl-N-oleyl-amide, L-Arginyl-β-alanine-N-palmityl-N-oleyl-amide, DOTAP (N-[1-(2,3-Dioleoyloxy)propyl]-N,N,N-trimethylammonium methyl-chloride), DOTMA (1,2-Di-O-octadecenyl-3-trimethylammonium propane (preferably as chloride salt)) and DC-cholesterol (3β-[N-(N′,N′-Dimethylaminoethane)-carbamoyl]cholesterol) wherein the neutral lipid is

a zwitterionic phospholipid selected from the group comprising Diphytanoyl-PE (1,2-Diphytanoyl-sn-glycero-3-phosphoethanolamine), DOPE (1,2-Dioleoyl-sn-glycero-3-phosphoethanolamine), DLPE (1,2-Dilauroyl-sn-glycero-3-phosphoethanolamine), DMPE ((1,2-Dimyristoyl-sn-glycero-3-phosphoethanolamine)), POPE ((1-Palmitoyl-2-oleoyl-sn-glycero-3-phosphoethanolamine)) and DSPE (1,2-Distearoyl-sn-glycero-3-phosphoethanolamine), or
an uncharged sterol lipid selected from a group comprising cholesterol and stigmasterol, and
wherein the shielding lipid is a PEGylated lipid selected from the group comprising methoxyPEG-DSPE ((1,2-Distearoyl-sn-glycero-3-phosphoethanolamine-N-[methoxy(polyethylene glycol)] (preferably as ammonium or sodium salt), methoxyPEG-DLG (1,2-Dilauroyl-rac-glycero-3-methoxypolyethylene glycol), methoxyPEG-DMG (1,2-Dimyristoyl-rac-glycero-3-methoxypolyethylene glycol), methoxyPEG-DPG (1,2-Dipalmitoyl-rac-glycero-3-methoxypolyethylene glycol), methoxyPEG-DSG (1,2-Distearoyl-rac-glycero-3-methoxypolyethylene glycol), methoxyPEG-c-DMA (N-[(Methoxy poly(ethylene glycol))carbamyl]-1,2-dimyristyloxlpropyl-3-amine), methoxyPEG-C8-ceramide (N-Octanoyl-sphingosine-1-{succinyl[methoxy(polyethylene glycol)]}) and methoxy PEG-C16-ceramide (N-Palmitoyl-sphingosine-1-{succinyl[methoxy (polyethylene glycol)]}), and
wherein the lipid composition forms particles, wherein the particle size is (a) from 30 nm to 200 nm, preferably from about 40 nm to about 140 nm, and even more preferable from about 60 nm to 120 nm or (b) from about 30 nm to about 100 nm, preferably from about 30 nm to about 60 nm.

Embodiment 67. The composition of Embodiment 66, wherein the composition comprises an mRNA.

Embodiment 68. The composition of Embodiment 65, wherein the lipid composition forms particles, wherein the particle size is from about 30 nm to about 200 nm, preferably the particle size is from about 40 nm to about 140 nm, and even more preferable from about 60 nm to 120 nm or (b) from about 30 nm to about 100 nm, preferably from about 30 nm to about 60 nm.

Embodiment 69. The composition of any one of Embodiments 66 to 68, wherein the particles comprise an mRNA.

Embodiment 70. The composition of any one of Embodiments 65 to 69, wherein the particle size is determined by dynamic light scattering.

Embodiment 71. The composition of any one of Embodiments 1 to 70, wherein the zeta potential of the particles is about +0 mV to about +80 mV, preferably the zeta potential of the particles is about +0 mV to about +45 mV.

Embodiment 72. The composition of Embodiment 71, wherein the zeta potential is measured by Laser Doppler Electrophoresis measurement.

Embodiment 73. The composition of any one of Embodiments 65 and 67 to 72, wherein the mRNA forms part of the particles.

Embodiment 74. The composition of any one of Embodiments 65 and 66 to 73, wherein the ratio of the mass of the total lipid content of the composition to the mass of the mRNA of the composition is from about 2 to about 50, preferably from about 5 to 40, and more preferably from about 10 to about 30.

Embodiment 75. The composition of any one of Embodiments 65 to 74, wherein the composition is suitable for delivering a therapeutically active agent to an endothelial cell.

Embodiment 76. The composition of Embodiment 75, wherein the endothelial cell is a pulmonary endothelial cell.

Embodiment 77. The composition of any one of Embodiments 65 to 76, wherein the composition is suitable for delivering a therapeutically active agent to vasculature, preferably pulmonary vasculature, more preferably to human pulmonary vasculature.

Embodiment 78. The composition of any one of Embodiments 65 to 77, wherein the therapeutically active agent is an mRNA, preferably the therapeutically active agent is the mRNA comprised by the composition.

Embodiment 79. The composition of any one of Embodiments 65 to 78, wherein the mRNA forms a complex with the lipid composition.

Embodiment 80. The composition of any one of Embodiments 65 to 79, wherein the composition comprises a carrier, preferably a pharmaceutically acceptable carrier.

Embodiment 81. The composition of Embodiment 80, wherein the carrier is selected from the group comprising water, an aqueous solution, preferably an isotonic aqueous solution, a sucrose solution, preferably an isotonic sucrose solution, a salt solution, preferably an isotonic salt solution, a buffer solution, preferably an isotonic buffer solution and a water miscible solvent.

Embodiment 82. The composition of Embodiment 81, wherein the carrier is selected from the group comprising an isotonic aqueous solution, an isotonic aqueous solution, an isotonic sucrose solution, an isotonic salt solution and an isotonic buffer solution.

Embodiment 83. The composition of any one of Embodiments 80 to 82, wherein the carrier is an aqueous sucrose solution, preferably a 270 mM aqueous sucrose solution or a 270 mM sucrose solution in a buffer, preferably the buffer is a 10 mM TRIS buffer pH 7.4.

Embodiment 84. The composition of any one of Embodiments 65 to 83, wherein the molecular weight of the PEG of the PEGylated lipid is from about 750 Da to about 5000 Da, preferably from about 1500 Da to 3000 Da.

Embodiment 85. The composition of any one of Embodiments 65 to 84, wherein the molar ratio of the lipids in the lipid composition is
from about 20 mol-% to about 80 mol-% of the cationic lipid,
from about 10 mol-% to about 70 mol-% of the neutral lipid, and
from about 0.1 mol-% to 10 mol-% of the PEGylated lipid,

wherein the overall lipid content is 100%.

Embodiment 86. The composition of Embodiment 85, wherein the molar ratio of the lipids in the lipid composition is
from about 35 mol-% to about 65 mol-% of the cationic lipid,
from about 35 mol-% to about 65 mol-% of the neutral lipid, and
from about 0.1 mol-% to 5 mol-% of the PEGylated lipid,
   wherein the overall lipid content is 100%.

Embodiment 87. The composition of any one of Embodiments 65 to 86, wherein cationic lipid is β-(L-Arginyl)-L-2,3-diamino propionic acid-N-palmityl-N-oleyl-amide or L-Arginyl-P-alanine-N-palmityl-N-oleyl-amide.

Embodiment 88. The composition of any one of Embodiments 65 to 87, preferably Embodiment 84, wherein the neutral lipid is the phospholipid Diphytanoyl-PE.

Embodiment 89. The composition of any one of Embodiments 65 to 88, preferably any one of Embodiments 87 and 88, more preferably Embodiment 88, wherein the PEGylated lipid is methoxyPEG2000-DSPE.

Embodiment 90. The composition of any one of Embodiments 65 to 89, wherein the molar ratio of the lipid composition is
50 mol-% of the cationic lipid, wherein the cationic lipid is β-(L-Arginyl)-L-2,3-diamino propionic acid-N-palmityl-N-oleyl-amide or L-Arginyl-β-alanine-N-palmityl-N-oleyl-amide,
49 mol-% of the neutral lipid, wherein the neutral lipid is Diphytanoyl-PE (1,2-Diphytanoyl-sn-glycero-3-phosphoethanolamine), and
1 mol-% of the shielding lipid, wherein the shielding lipid is mPEG-2000-DSPE (1,2-distearoyl-sn-glycero-3-phosphoethanolamine-N-[methoxy(polyethylene glycol)-2000] (preferably as sodium salt)).

Embodiment 91. The composition of any one of Embodiments 1 to 90 for use in a method for the treatment and/or prevention of a disease.

Embodiment 92. The composition for use of Embodiment 91, wherein the treatment and/or prevention comprises administration of the composition or the mRNA of the composition to an endothelial cell, preferably an endothelial cell of vasculature, more preferably an endothelial cell of lung vasculature.

Embodiment 93. The composition for use of Embodiment 92, wherein the endothelium is human endothelium.

Embodiment 94. The composition for use of any one of Embodiments 92 to 93, wherein the disease is a disease where a target molecule involved in the pathological mechanism underlying the disease is present in an endothelial cell of lung vasculature and the provision of the mRNA provides for a therapeutic effect.

Embodiment 95. The composition for use of any one of Embodiments 91 to 94, wherein the disease is selected from the group comprising acute respiratory distress syndrome, acute lung injury, lung cancer, pulmonary metastasis, pulmonary hypertension and pulmonary artery hypertension.

Embodiment 96. The composition of any one of Embodiments 91 to 95, wherein the composition is for intravenous, intramuscular, subcutaneous, intradermal, intravitreal, intrathecal, perivascular, intranasal administration, and/or for administration by inhalation, preferably, the composition is for intravenous administration.

Embodiment 97. Use of a composition of any one of Embodiments 1 to 90, in the manufacture of a medicament for the treatment and/or prevention of a disease.

Embodiment 98. Use of Embodiment 97, wherein treatment and/or prevention of a disease comprises administration of the composition or of the nucleic acid molecule, preferably the mRNA molecule of the composition to an endothelial cell, preferably an endothelial cell of vasculature, more preferably an endothelial cells of lung vasculature.

Embodiment 99. Use of Embodiment 98, wherein the endothelium is human endothelium.

Embodiment 100. Use of any one of Embodiments 98 to 99, wherein the disease is a disease where a target molecule involved in the pathological mechanism underlying the disease is present in an endothelial cell of lung vasculature and the provision of the nucleic acid molecule, preferably of mRNA molecule provides for a therapeutic effect.

Embodiment 101. Use of any one of Embodiments 97 to 100, wherein the disease is selected from the group comprising acute respiratory distress syndrome, acute lung injury, lung cancer, pulmonary metastasis, pulmonary hypertension and pulmonary artery hypertension.

Embodiment 102. Use of any one of Embodiments 97 to 101, wherein the medicament is for intravenous, intramuscular, subcutaneous, intradermal, intravitreal, intrathecal, perivascular, intranasal administration, and/or for administration by inhalation, preferably the medicament is for intravenous administration.

Embodiment 103. A pharmaceutical composition comprising a composition of any one of Embodiments 1 to 90 and a pharmaceutically active agent.

Embodiment 104. The pharmaceutical composition of Embodiment 103, wherein the nucleic acid molecule and preferably the mRNA of the composition of any one of Embodiments 1 to 90 is a or the therapeutically active agent.

Embodiment 105. The pharmaceutical composition of any one of Embodiments 103 to 104, for use in the treatment and/or prevention of a disease, wherein the disease is preferably selected from the group comprising acute respiratory distress syndrome, acute lung injury, lung cancer, pulmonary metastasis, pulmonary hypertension and pulmonary artery hypertension.

Embodiment 106. A kit comprising a composition of any one of Embodiments 1 to 90 and instructions of use.

Embodiment 107. A method for the treatment and/or prevention of a disease, wherein the method comprises administering to a subject in need thereof a composition of any one of Embodiments 1 to 90, wherein the nucleic acid molecule, preferably the mRNA molecule of the composition is therapeutically active, more preferably, the nucleic acid molecule, more preferably mRNA molecule is suitable for the treatment of the disease.

Embodiment 108.The method of Embodiments 107, wherein the disease is preferably selected from the group comprising acute respiratory distress syndrome, acute lung injury, lung cancer, pulmonary metastasis, pulmonary hypertension and pulmonary artery hypertension.

Embodiment 109. The method of any one of Embodiments 107 to 108, wherein the subject is selected from the group comprising man, mouse, rat, rabbit, hamster, guinea pig, monkey, dog, cat, pig, sheep, goat, cow and horse, preferably the subject is man.

Embodiment 110. A method for preparing a composition of any one of Embodiments 1 to 90, wherein the method comprises mixing a solution comprising the lipid components of the lipid composition with a solution comprising a nucleic acid molecule, preferably an mRNA molecule, wherein more preferably the mixing is an in-line mixing.

Embodiment 111. The method of Embodiment 111, wherein the mixing is a non-turbulent and diffusion-based mixing, preferably a rapid non-turbulent and diffusion-based mixing.

Embodiment 112. The method of any one of Embodiments 110 to 111, wherein the mixing is a microfluidic mixing, preferably a microfluidic mixing using a microfluidic mixing device.

Embodiment 113. The method of Embodiment 112, wherein the microfluidic mixing device is selected from the group comprising a staggered herringbone mixer, a microfluidic hydrodynamic mixing device or a Dean Vortex bifurcating mixing device.

Embodiment 114. The method of any one of Embodiments 110 to 113, wherein the solution comprising the lipid component comprises the components of the lipid composition in a water miscible organic solvent.

Embodiment 115. The method of Embodiment 114, wherein the water miscible organic solvent is selected from the group comprising ethanol, acetone, 1-butanol, 2-butanol, tert.-butanol, 3-methyl-1-butanol, 2-methyl-1-propanol, 1-propanol, 2-propanol, dimethylsulfoxide, preferably the water miscible solvent is selected from the group comprising ethanol, tert.-butanol and 1-butanol.

Embodiment 116. The method of any one of Embodiments 110 to 115, wherein the solution comprising the nucleic acid molecule, preferably the mRNA molecule comprises the nucleic acid molecule, preferably the mRNA molecule, in water or an aqueous buffer.

Embodiment 117. The method of Embodiment 116, wherein

(a) if the composition is a composition any one of Embodiments 1 to 64, the aqueous buffer is a buffer of the tricarboxylic acid; and
(b) if the composition is a composition of any one of Embodiment 65 to 90, the buffer is acetate buffer, TRIS buffer (2-Amino-2-(hydroxymethyl)propane-1,3-diol) or HEPES buffer (2-[4-(2-hydroxyethyl)piperazin-1-yl] ethanesulfonic acid).

Embodiment 118. The method of Embodiment 117, wherein the buffer is a buffer selected from the group comprising a buffer of citric acid, of isocitric acid (1-hydroxypropane-1,2,3-tricarboxylic acid), of cis-aconitic acid, of trans-aconitic acid, of a mixture of both cis-aconitic acid and trans-aconitic acid, of propane-1,2,3-tricarboxylic acid (tricarballylic acid), of agaric acid, of trimesic acid, and of any mixture thereof, preferably a citrate buffer.

Embodiment 119. The method of any one of Embodiments 117 to118, wherein the buffer is a buffer of the tricarboxylic acid, preferably a citrate buffer, wherein the buffer is from 0.1 mM to 300 mM, preferably from 1 to 150 mM, and more preferably from 10 mM to 80 mM.

Embodiment 120. The method of any one of Embodiments 117 to 119, wherein the buffer is a buffer of the tricarboxylic acid, preferably a citrate buffer, wherein the pH of the buffer is from 3 to 7, preferably from 4 to 6, and more preferably from 4.5 to 5.7.

Embodiment 121. The method of any one of Embodiments 110 to 120, wherein a volumetric mixing ratio of the solution comprising the nucleic acid molecule, preferably the mRNA molecule, to the solution comprising the lipid components of the lipid composition is from about 1:1 to about 6:1, preferably from about 2:1 to about 4:1.

Embodiment 122. The method of any one of Embodiments 110 to 121, wherein the organic solvent is removed from the mixture after the mixing.

Embodiment 123.

The method of Embodiment 122, wherein the organic solvent is removed from the mixture after the mixing by dialysis, tangential flow filtration and/or diafiltration.

Embodiment 124. The method of Embodiment 123, wherein the dialysis, tangential flow filtration and/or diafiltration removes less than 50%, less than 45%, less than 40%, less than 35%, less than 30%, less than 25%, less than 20%, less than 15%, less than 10%, less than 5%, less than 1% or 0% of the tricarboxylic acid from the composition, preferably from the lipid composition, more preferably from the particles formed by the lipid composition.

Embodiment 125. The method of any one of Embodiments 123 to 124, wherein the ultrafiltration membrane used in tangential flow filtration, diafiltration and/or dialysis has a molecular weight cut-off of from about 1,500 Da to about 500,000 Da, preferably of from about 1,500 Da to about 100,000 Da and more preferably of from about 1,500 Da to about 30,000 Da.

Embodiment 126. The method of any one of Embodiments 110 to 125, wherein the reaction mixture is concentrated after the mixing, preferably after removing the organic solvent.

Embodiment 127. The method of Embodiment 126, wherein the reaction mixture is concentrated by tangential flow filtration and/or diafiltration.

Embodiment 128.

The method of any one of Embodiments 126 to 127, wherein the reaction mixture after concentration is a ready-to-use composition.

In accordance with the present invention, the problem underlying the present invention is solved by a composition comprising a lipid composition, a tricarboxylic acid and an mRNA, wherein the lipid composition comprises a cationic lipid, a neutral lipid and a shielding lipid; this composition which is, among others, subject to Embodiment 1 as recited above, is also referred to as the first aspect of the (present) invention.

In accordance with the present invention, the problem underlying the present invention is also solved by a composition comprising a lipid composition, wherein the lipid composition comprises a cationic lipid, a neutral lipid and a shielding lipid, wherein the cationic lipid is selected from the group comprising β-(L-Arginyl)-L-2,3-diamino propionic acid-N-palmityl-N-oleyl-amide, L-Arginyl-β-alanine-N-palmityl-N-oleyl-amide, DOTAP (N-[1-(2,3-Dioleoyloxy)propyl]-N,N,N-trimethylammonium methyl-chloride), DOTMA (1,2-Di-O-octadecenyl-3-trimethylammonium propane (preferably as chloride salt)) and DC-cholesterol (3β-[N-(N′,N′-Dimethylaminoethane)-carbamoyl]cholesterol), wherein the neutral lipid is a zwitterionic phospholipid selected from the group comprising Diphytanoyl-PE (1,2-Diphytanoyl-sn-glycero-3-phosphoethanolamine), DOPE (1,2-Dioleoyl-sn-glycero-3-phosphoethanol amine), DLPE (1,2-Dilauroyl-sn-glycero-3-phosphoethanolamine), DMPE ((1,2-Dimyristoyl-sn-glycero-3-phosphoethanolamine)), POPE ((1-Palmitoyl-2-oleoyl-sn-glycero-3-phosphoethanolamine)) and DSPE (1,2-Distearoyl-sn-glycero-3-phospho), or

an uncharged sterol lipid selected from a group comprising cholesterol and stigmasterol,
wherein the shielding lipid is a PEGylated lipid selected from the group comprising methoxyPEG-DSPE ((1,2-Distearoyl-sn-glycero-3-phosphoethanolamine-N-[methoxy(polyethylene glycol)] (preferably as ammonium or sodium salt), methoxyPEG-DLG (1,2-Dilauroyl-rac-glycero-3-methoxypolyethylene glycol), methoxyPEG-DMG (1,2-Dimyristoyl-rac-glycero-3-methoxypolyethylene glycol), methoxyPEG-DPG (1,2-Dipalmitoyl-rac-glycero-3-methoxypolyethylene glycol), methoxyPEG-DSG (1,2-Distearoyl-rac-glycero-3-methoxypolyethylene glycol), methoxyPEG-c-DMA (N-[(Methoxy poly(ethylene glycol))carbamyl]-1,2-dimyristyloxlpropyl-3-amine), methoxyPEG-C8-ceramide (N-Octanoyl-sphingosine-1-{succinyl[methoxy (polyethylene glycol)]}), methoxy PEG-C16-ceramide (N-Palmitoyl-sphingosine-1-{succinyl[methoxy (polyethylene glycol)]}), and wherein the composition comprises an mRNA; this composition which is, among others, subject to Embodiment 65 as recited above, is also referred to as the second aspect of the (present) invention.

In accordance with the present invention, the problem underlying the present invention is also solved by a composition comprising a lipid composition, wherein the lipid composition comprises a cationic lipid, a neutral lipid and a shielding lipid, wherein the cationic lipid is selected from the group comprising β-(L-Arginyl)-L-2,3-diamino propionic acid-N-palmityl-N-oleyl-amide, L-Arginyl-β-alanine-N-palmityl-N-oleyl-amide, DOTAP (N-[1-(2,3-Dioleoyloxy)propyl]-N,N,N-trimethylammonium methyl-chloride), DOTMA (1,2-Di-O-octadecenyl-3-trimethylammonium propane (preferably as chloride salt)) and DC-cholesterol (3β-[N-(N′,N′-Dimethylaminoethane)-carbamoyl]cholesterol) wherein the neutral lipid is a zwitterionic phospholipid selected from the group comprising Diphytanoyl-PE (1,2-Diphytanoyl-sn-glycero-3-phosphoethanolamine), DOPE (1,2-Dioleoyl-sn-glycero-3-phosphoethanolamine), DLPE (1,2-Dilauroyl-sn-glycero-3-phosphoethanolamine), DMPE ((1,2-Dimyristoyl-sn-glycero-3-phosphoethanolamine)), POPE ((1-Palmitoyl-2-oleoyl-sn-glycero-3-phosphoethanolamine)) and DSPE (1,2-Distearoyl-sn-glycero-3-phosphoethanolamine), or

an uncharged sterol lipid selected from a group comprising cholesterol and stigmasterol, and
wherein the shielding lipid is a PEGylated lipid selected from the group comprising methoxyPEG-DSPE ((1,2-Distearoyl-sn-glycero-3-phosphoethanol amine-N-[methoxy(polyethylene glycol)] (preferably as ammonium or sodium salt), methoxyPEG-DLG (1,2-Dilauroyl-rac-glycero-3-methoxypolyethylene glycol), methoxyPEG-DMG (1,2-Dimyristoyl-rac-glycero-3-methoxypoly ethylene glycol), methoxy PEG-DPG (1,2-Dipalmitoyl-rac-glycero-3-methoxypolyethylene glycol), methoxyPEG-DSG (1,2-Distearoyl-rac-glycero-3-methoxypolyethylene glycol), methoxyPEG-c-DMA (N-[(Methoxy poly(ethylene glycol))carbamyl]-1,2-dimyristyloxlpropyl-3-amine), methoxyPEG-C8-ceramide (N-Octanoyl-sphingosine-1-{succinyl[methoxy(poly ethylene glycol)]}) and methoxy PEG-C16-ceramide (N-Palmitoyl-sphingosine-1-{succinyl[methoxy(polyethylene glycol)]}), and
and wherein the lipid composition forms particles, wherein the particle size is (a) from 30 nm to 200 nm, preferably from about 40 nm to about 140 nm, more preferably from about 60 nm to about 120 nm; or (b) from about 30 nm to about 100 nm, preferably from about 30 nm to about 60 nm; this composition which is, among others, subject to Embodiment 66 as recited above, is also referred to as the third aspect of the (present) invention.

In accordance with the present invention, the problem underlying the present invention is solved by a composition according to the first, second and third aspect, including any embodiment thereof, for use in a method for the treatment and/or prevention of a disease; such composition for use which is, among others, subject to Embodiment 91 as recited above, is also referred to as the fourth aspect of the (present) invention.

It is to be acknowledged that any embodiment of the first aspect of the present invention is equally an embodiment of the second and third embodiment of the present invention and vice versa, as long as not indicated to the contrary.

In accordance with the present invention, the problem underlying the present invention is solved by the use of a composition of the first, second and third aspect, including any embodiment thereof, in the manufacture of a medicament for the treatment and/or prevention of a disease; this use which is, among others, subject to Embodiment 97 as recited above, is also referred to as the fifth aspect of the (present) invention.

In accordance with the present invention, the problem underlying the present invention is solved by a pharmaceutical composition comprising a composition according to the first, second and third aspect, including any embodiment thereof, and a pharmaceutically active agent; this pharmaceutical composition which is, among others, subject to Embodiment 103 as recited above, is also referred to as the sixth aspect of the (present) invention. The pharmaceutical composition according to the sixth aspect is also a pharmaceutical composition comprising a composition according to the first, second and third aspect, including any embodiment thereof, wherein the nucleic acid, preferably the mRNA, is the therapeutic agent of the pharmaceutical composition and the remainder of such composition according to the first, second and third aspect, including any embodiment thereof, or a part thereof, forms the or a pharmaceutically acceptable excipient contained in the pharmaceutical composition. Preferably the remainder of such composition according to the first, second and third aspect, including any embodiment thereof except the lipid composition, preferably the lipids thereof, forms the or a pharmaceutically acceptable excipient contained in the pharmaceutical composition.

In accordance with the present invention, the problem underlying the present invention is solved by a kit comprising a composition according to the first, second and third aspect, including any embodiment thereof, and instructions of use; this kit which is, among others, subject to Embodiment 106 as recited above, is also referred to as the seventh aspect of the (present) invention.

In accordance with the present invention, the problem underlying the present invention is solved by a method for the treatment and/or prevention of a disease, wherein the method comprises administering to a subject in need thereof a composition according to the first, second and third aspect, including any embodiment thereof, wherein the nucleic acid molecule, preferably the mRNA, of the composition is therapeutically active, preferably the nucleic acid molecule, more preferably the mRNA is suitable for the treatment of the disease; this method which is, among others, subject to Embodiment 107 as recited above, is also referred to as the eighth aspect of the (present) invention.

In accordance with the present invention, the problem underlying the present invention is solved by a method for preparing a composition according to the first, second and third aspect, including any embodiment thereof, wherein the method comprises mixing a solution comprising the lipid components of the lipid composition with a solution comprising the nucleic acid molecule, preferably the mRNA molecule, wherein preferably the mixing is an in-line mixing; this method which is, among others, subject to Embodiment 110 as recited above, is also referred to as the ninth aspect of the (present) invention.

In accordance with the present invention, it is referred to the composition according to the first, second and third aspect also as the composition of the (present) invention or the composition according to the (present) invention.

It is to be acknowledged that if it is referred, particularly in connection with the first aspect of the present invention, to the lipid nanoparticles of the prior art, such lipid nanoparticles of the prior art are lipid nanoparticles which do not comprise a tricarboxylic acid as the composition and lipid nanoparticles of the present invention.

The present invention and the first aspect thereof in particular, including any embodiment thereof, is based on the surprising finding that a composition comprising a lipid composition and a tricarboxylic acid such as citric acid provides for an increased number of lipid particles formed from the lipid composition at a given constant amount of said nucleic acid molecule while concomitantly maintaining a monodisperse particle size distribution. The latter even more so if the composition comprises a nucleic acid molecule such as an mRNA. In other words, if a given amount of the lipid composition and a given amount of a nucleic acid is provided so as to form a composition comprising lipid particles and lipid nanoparticles (LNPs) in particular, the tricarboxylic acid allows an even distribution of the nucleic acid molecule over the formed lipid particles which, in addition, show a monodisperse particle size distribution, even at a molar mass ratio of total lipids to nucleic acid molecule where, in the absence of the tricarboxylic acid, the composition would show a polydisperse particle size distribution. The lipid nanoparticles thus generated typically have an increased mass ratio, wherein mass ratio as used herein is defined as mass of total lipids of the composition to mass of nucleic acid, and mRNA in particular, in the composition (e.g. m/m ratio (total lipids/mRNA)). The above finding is in particular observed in case the amount of and, therefore, the number of negative charges of the nucleic acid molecules is lower, preferably significantly lower, than the overall number of positive charges of the lipids in the composition. Such scenario is sometimes also referred to as the amount of the nucleic acid molecules being limiting in the composition.

Without wishing to be bound by any theory, the present inventors assume that the tricarboxylic acid acts, and can be used insofar, as a stuffer molecule in order to homogenously distribute a given—limiting—amount of a nucleic acid molecules over a high number of lipid particles and lipid nanoparticles in particular. This is in particular advantageous if the tricarboxylic acid is non-toxic and/or non-immunogenic.

The present inventors also surprisingly found that adding a tricarboxylic acid to a composition comprising a lipid composition and a nucleic acid molecule, wherein the lipid composition comprises a cationic lipid, a neutral lipid and a shielding lipid and wherein the composition shows a positive charge excess arising from a larger number of positive charges provided by the cationic lipid molecules in the composition compared to the smaller number of negative charges provided by the nucleic acid molecules in the composition and the shielding lipid, if any, avoid the transition of the composition from one having a monodisperse particle distribution to one having a polydisperse particle distribution.

In an embodiment, the content of the tricarboxylic acid and citric acid in particular in the composition of the invention can be determined by means of the assay disclosed in Example 6 the disclosure of which is incorporated in the general part of the description by reference.

In an embodiment of the first, second and third aspect of the present invention, including any embodiment thereof and as preferably used herein, a charge is an electrical charge. In accordance therewith, a negative charge is a negative electrical charge, and a positive charge is a positive electrical charge.

In connection with the monodisperse particle size distribution of the composition of the present invention it is to be acknowledged that such monodisperse particle size distribution is advantageous over a polydisperse particle size distribution. Such advantage resides in particular in a more homogenous distribution of the composition and the particles of such composition in particular, in a subject upon administration to the subject, preferably administration to the subject for therapeutic, diagnostic and/or theragnostic purpose. Such more homogenous distribution in the subject allows a more controlled and more effective transfection of target cells and thus increased efficacy in the delivery of the payload of the composition and particles, respectively, such as a nucleic acid and mRNA in particular. Typically, the more controlled and more effective transfection of target cells in a subject go along with less side effects in the subject. Furthermore, a more homogenous particle distribution allows a more reliable batch-to-batch comparability within the nanoparticle manufacturing process.

The present invention and the first aspect thereof in particular, including any embodiment thereof, is based on the further surprising finding that the use of a tricarboxylic acid such as citric acid in a lipid composition provides for an increased release of a nucleic acid molecule from such tricarboxylic acid comprising lipid composition upon transfection of a cell with said composition. Without wishing to be bound by any theory, the present inventors assume that such increased release of the nucleic acid molecule results from the high buffer capacity of the tricarboxylic acid at the endosomal pH, which is typically about 6. Due to such high buffer capacity, endosomal acidification and thus endosomal maturation can be prevented and, as a consequence the endosomal release of the intact nucleic acid molecule into the cytoplasma is increased. Such increased release of an intact nucleic acid molecule goes along a faster and more comprehensive effect, such as a therapeutic effect, of the nucleic acid molecule, if the nucleic acid molecule is a therapeutically active molecule.

The present inventors have surprisingly found that a composition comprising a lipid composition, wherein the lipid composition comprises a cationic lipid, a neutral lipid and a shielding lipid,

wherein the cationic lipid is selected from the group comprising β-(L-Arginyl)-L-2,3-diamino propionic acid-N-palmityl-N-oleyl-amide, L-Arginyl-β-alanine-N-palmityl-N-oleyl-amide, DOTAP (N-[1-(2,3-Dioleoyloxy)propyl]-N,N,N-trimethylammonium methyl-chloride), DOTMA (1,2-Di-O-octadecenyl-3-trimethylammonium propane (chloride salt)) and DC-cholesterol (3β-[N-(N′,N′-Dimethylaminoethane)-carbamoyl] cholesterol),
wherein the neutral lipid is
a zwitterionic phospholipid selected from the group comprising Diphytanoyl-PE (1,2-Diphytanoyl-sn-glycero-3-phosphoethanolamine), DOPE (1,2-Dioleoyl-sn-glycero-3-phosphoethanolamine), DLPE (1,2-Dilauroyl-sn-glycero-3-phosphoethanolamine), DMPE ((1,2-Dimyristoyl-sn-glycero-3-phosphoethanol amine)), POPE ((1-Palmitoyl-2-oleoyl-sn-glycero-3-phosphoethanolamine)) and DSPE (1,2-Distearoyl-sn-glycero-3-phosphoethanolamine), or
an uncharged sterol lipid selected from a group comprising cholesterol and stigmasterol,
wherein the shielding lipid is a PEGylated lipid selected from the group comprising methoxyPEG-DSPE ((1,2-Distearoyl-sn-glycero-3-phosphoethanolamine-N-[methoxy(polyethylene glycol)] (ammonium or sodium salt), methoxyPEG-DLG (1,2-Dilauroyl-rac-glycero-3-methoxypolyethylene glycol), methoxyPEG-DMG (1,2-Dimyristoyl-rac-glycero-3-methoxypolyethylene glycol), methoxyPEG-DPG (1,2-Dipalmitoyl-rac-glycero-3-methoxypolyethylene glycol), methoxyPEG-DSG (1,2-Distearoyl-rac-glycero-3-methoxypolyethylene glycol), methoxyPEG-c-DMA (N-[(Methoxy poly(ethylene glycol))carbamyl]-1,2-dimyristyloxlpropyl-3-amine), methoxyPEG-C8-ceramide (N-Octanoyl-sphingosine-1-{succinyl[methoxy(polyethylene glycol)]}), methoxyPEG-C16-ceramide (N-Palmitoyl-sphingosine-1-{succinyl[methoxy(poly ethylene glycol)]}) is particularly effective in targeting an endothelial cell, preferably an endothelial cell of vasculature and more preferably an endothelial cell of lung vasculature.

In connection with the endothelium formed by endothelial cells of the lung vasvculature is acknowledged by a person skilled in the art that the vascular endothelium is the inner-most structure that coats the interior walls of arteries, capillaries and veins. Endothelial cells (EC) were described to anchor to an 80-nm-thick basal lamina (BL). Both EC and BL constitute the vascular intima, establishing a hemocompatible surface, estimated a total combined surface area of 3000-6000 m2 in the human body, comprising 1 to 6×1013 EC (Krüger-Genge, A., Blocki, A., Franke, R.-P. & Jung, F. Vascular Endothelial Cell Biology: An Update. Int J Mol Sci 20, 4411 (2019).)

Additionally, the present inventors have surprisingly found that such targeting allows delivery of mRNA to said endothelial cell, whereby such mRNA is part of the composition of the present invention, preferably such mRNA is associated with the lipid composition of the composition of the invention. More preferably, such mRNA is part of particles formed by the lipid composition of the composition of the present invention.

Again, without wishing to be bound by any theory, the present inventors assume that said delivery of mRNA to endothelial cells is, in part, caused by the size distribution of the particles formed by the lipid composition of the composition of the present invention.

The above finding is insofar surprising as it contrasts typical systemic behavior of cationic lipid nano-complexes which generally show prevalent gene knockdown in liver tissue with only transient accumulation of siRNA in the lung (Polach, K J et al. (2012), Mol Ther 20: 91-100; Schroeder, A et al. (2010), J Intern Med 267: 9-21; Tao, W et al. (2010), Mol Ther 18: 1657-1666).

As preferably used herein, a delivery agent or a delivery vehicle is a composition comprising the lipid composition of the invention. As also preferably used herein, a delivery agent or a delivery vehicle is a composition of the invention. A delivery agent or a delivery vehicle as preferably used herein is an agent or a vehicle such as a composition which is suitable to deliver a compound to a structure; preferably such structure is an organ, tissue or cell; more preferably such structure is an organ, tissue or cell. In a preferred embodiment such compound is a therapeutically active agent, a biologically active agent or a pharmaceutically active agent.

As preferably used herein, a therapeutically active agent is a compound which is suitable to elicit in a host organism a therapeutic or therapeutically beneficial effect. In a preferred embodiment, the therapeutically active agent is a nucleic acid molecule and more preferably an mRNA.

In an embodiment of each and any aspect of the present invention, including any embodiment thereof, the nucleic acid molecule is an mRNA molecule.

In an embodiment of the first, second and third aspect of the present invention, including any embodiment thereof, the tricarboxylic acid is bound in or bound to the particles formed by the lipid composition in the composition of the invention. Such binding is stable; more specifically the thus bound tricarboxylic acid is non-removably by means of dialysis. Conditions for such dialysis are preferably as follows: The composition of the present invention and the lipid composition of the present invention, respectively, may be, in an embodiment, dialyzed using a dialysis membrane having a MW cut-off of 3.5 kD against 10 mM TRIS/9% sucrose. Preferably, such dialysis is carried out until all of the undesired components are removed to the extent possible under such dialysis conditions. In an alternative embodiment, the composition of the present invention and the lipid composition of the present invention, respectively, may be dialyzed using a standard dialysis cassette under the following conditions: A sample containing the composition of the present invention and the lipid composition of the present invention, respectively, is dialyzed in a floating dialysis cassette for 2 hours at room temperature or 4° C. while gently stirring the dialysis buffer; the dialysis buffer is changed and dialysis is continued for another 2 hours; subsequently, the dialysis buffer is changed and dialysis continued overnight; all single dialysis steps shout be carried out at room temperature or 4° C.; during the course of the dialysis procedure a total of dialysis buffer of at least 300 times the volume of the sample should be used. Preferably, the purpose of such dialysis step is the removal of the solvent(s) used in the preparation of the lipid composition. It will be acknowledged by a person skilled in the art that other dialysis conditions might be used for the same purpose and more particularly for achieving the same result, in particular the removal of the solvent(s) used in the preparation of the lipid composition.

In an embodiment of the first, second and third aspect of the present invention, including any embodiment thereof, the lipid composition comprises, at least, in terms of lipid species a cationic lipid, a neutral lipid and a shielding lipid. It will be understood by a person skilled in the art that the lipid composition of the composition of the invention contains a plurality of individual lipid molecules of each of said three lipid species.

It is within the present invention and insofar an embodiment of each and any aspect of the present invention, including any embodiment thereof, that the lipid composition comprises two or more different species of a neutral lipid.

It is within the present invention and insofar an embodiment of each and any aspect of the present invention, including any embodiment thereof, that the lipid composition comprises two or more different species of a cationic lipid.

It is within the present invention and insofar an embodiment of each and any aspect of the present invention, including any embodiment thereof, that the lipid composition comprises two or more different species of a shielding lipid.

It is within the present invention and insofar an embodiment of each and any aspect of the present invention, including any embodiment thereof, that the composition comprises two or more different species of a nucleic acid molecule.

In an embodiment of the present invention defined by the various aspects, including any embodiment thereof, the term nucleic acid and nucleic acid molecule are used interchangeably.

In an embodiment of the present invention defined by the various aspects, including any embodiment thereof, the term mRNA and mRNA molecule are used interchangeably.

In an embodiment of the present invention defined by the various aspects, including any embodiment thereof, if reference is made, particularly in connection with any mass ratio, to total lipids, total lipids is meant to refer to the sum of the mass of all of the lipids of the lipid composition and, respectively, lipids contained in the composition of the present invention.

In an embodiment of the present invention defined by the various aspects, including any embodiment thereof, if reference is made, particularly in connection with any mass ratio to the mass of a nucleic acid or a nucleic acid molecule contained in a composition of the present invention, the mass of the nucleic acid and of the nucleic acid molecule, respectively, is the sum of all the mass of the entire nucleic acid and, respectively, the sum of all the mass of the nucleic acid molecules contained in the composition of the present invention.

In an embodiment of the present invention defined by the various aspects, including any embodiment thereof, if reference is made, particularly in connection with any mass ratio to the mass of an mRNA or an mRNA molecule contained in a composition of the present invention, the mass of the mRNA and of the mRNA molecule, respectively, is the sum of all the mass of the entire mRNA and, respectively, the sum of all the mass of the mRNA molecules contained in the composition of the present invention.

In an embodiment of the present invention defined by the various aspects, including any embodiment thereof, and as preferably used herein, the expression m/m ratio (total lipids/mRNA) is the same as m/m ratio (total lipids/mRNA molecules), as m/m ratio (total lipids/nucleic acid) and as m/m ratio (total lipids/nucleic acid molecules). The term m/m ratio (total lipids/mRNA) is thus preferably used in a generic manner, regardless whether the nucleic acid is an mRNA or any other nucleic acid and nucleic acid molecule, respectively. In a more preferred embodiment, however, the term m/m ratio (total lipids/mRNA) actually refers to the mass of the mRNA and mRNA molecules, respectively, contained in the composition of the present invention.

In an embodiment and as preferable used herein, a ready-to-use composition is a composition which can be immediately used for the intended purpose, preferably used for the intended purpose without having to apply any further measures to the composition which measures have or could have an impact on the composition's physical characteristics (e.g. solid state or liquid state), chemical characteristics (such as quantitative and/or qualitative chemical composition, purity or sterility) and activities and/or biological characteristics and activities (such as therapeutic efficacy). In a preferred embodiment the composition of the present invention is a ready-to-use composition for administration to a subject, preferably administration of therapeutic, diagnostic and/or theragnostic application.

In an embodiment of the method for preparing a composition according to the first, second and third aspect of the present invention, including any embodiment thereof, a ready-to-use composition is obtained. It is within the present invention that such ready-to-use composition is obtained after the one or more further reaction step such as the dialysis step and/or the diafiltration step. It is, however, also within the present invention that such ready-to-use composition is obtained after an or the another reaction step, preferably a concentration step carried out after the dialysis step and/or the diafiltration step.

It is within the present invention that the reaction mixture obtained from the mixing of a solution comprising the lipid components of the lipid composition with a solution comprising the mRNA in a buffer of a tricarboxylic acid is subjected to one or more further reaction steps. Preferably, such reaction step is a dialysis step or a diafiltration step. The purpose of such further reaction steps and the dialysis step and/or diafiltration step is to remove all organic solvent that were used for or in the preparation of the lipid composition of the composition of the present invention, to remove and replace the used aqueous buffer by a buffer that is appropriate for the intended use, and provides an appropriate pH and tonicity. It is within the present invention that the further reaction step and either the dialysis step, the diafiltration step or both are followed by another reaction step, preferably such another reaction step is a concentration step. The purpose of such concentration step is preferably to adjust the final concentration of the nucleic acid molecule and the mRNA, respectively, to a value appropriate for the intended application and use.

In connection with the instant invention, therapy and treatment, respectively, also encompasses prevention. In accordance therewith, a therapeutically active agent is, in an embodiment, also an agent which is active in prevention of a disease. In an alternative embodiment, a therapeutically active agent is not active in the prevention of a disease.

The composition of the present invention comprises a cationic lipid. It will be acknowledged by a person skilled in the art that any of the NH or NH2 group(s) present in said lipid are, preferably, present in a protonated form. Typically, any positive charge of said lipid is compensated by the presence of an anion. Such anion can be a monovalent or polyvalent anion. Preferred anions are halides, acetate and trifluoroacetate. Halides as used herein are preferably chlorides, fluorides, iodides and bromides. Most preferred are chlorides. Upon association of the cationic lipid and the therapeutically or pharmaceutically or biologically active compound to be transferred into a cell, the halide anion is replaced by the said active compound which preferably exhibits one or several negative charges, although it has to be acknowledged that the overall charge of the biologically active compound is not necessarily negative. The same considerations are equally applicable to the other compounds of the composition of the invention. In case such compound is an anionic compound or bears one or several negative charges such negative charges may be compensated by the presence of a cation. Such cation can be a monovalent or polyvalent cation. Preferred cations are ammonium, sodium or potassium.

The sterol compound of the composition of the invention can be either synthetic or be obtained from natural sources such as sheep wool or plants.

The PEGylated lipid of the composition of the invention is available from commercial sources such as NOF Corporation, Japan; Avanti Polar Lipids, US; or Cordon Pharma, Switzerland.

In an embodiment, compound (3-(L-Arginyl)-L-2,3-diamino propionic acid-N-palmityl-N-oleyl-amide) is of the following formula:

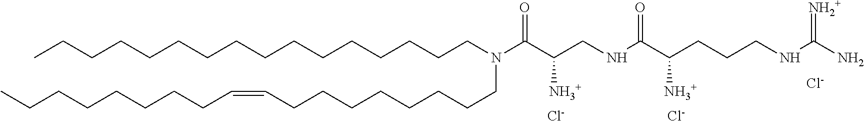 [SMILES: CCCCCCCC/C=C\CCCCCCCCN(CCCCCCCCCCCCCCCC)C(=O)[C@@H]([NH3+])CNC(=O)[C@@H]([NH3+])CCCNC(N)=[NH2+] ; [Cl-] ; [Cl-] ; [Cl-]]

In an embodiment, compound L-Arginyl-P-alanine-N-palmityl-N-oleyl-amide is of the following formula:

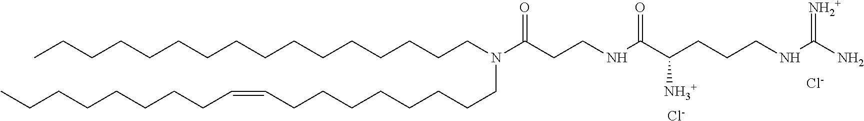 [SMILES: CCCCCCCC/C=C\CCCCCCCCN(CCCCCCCCCCCCCCCC)C(=O)CCNC(=O)[C@@H]([NH3+])CCCNC(N)=[NH2+] ; [Cl-] ; [Cl-]]

In an embodiment, compound DOTAP (N-[1-(2,3-Dioleoyloxy)propyl]-N,N,N-trimethylammonium methyl-chloride) is of the following formula:

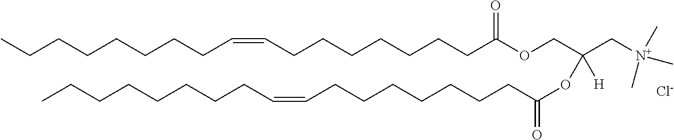 [SMILES: CCCCCCCC/C=C\CCCCCCCC(=O)OCC(C[N+](C)(C)C)OC(=O)CCCCCCC/C=C\CCCCCCCC ; [Cl-]]

In an embodiment, compound DOTMA (1,2-Di-O-octadecenyl-3-trimethylammonium propane (preferably as chloride salt)) is of the following formula:

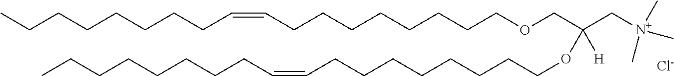 [SMILES: CCCCCCCC/C=C\CCCCCCCCOCC(C[N+](C)(C)C)OCCCCCCCC/C=C\CCCCCCCC ; [Cl-]]

In an embodiment, compound DC-Cholesterol (3β-[N-(N′,N′-Dimethylaminoethane)-carbamoyl]cholesterol hydrochloride) is of the following formula:

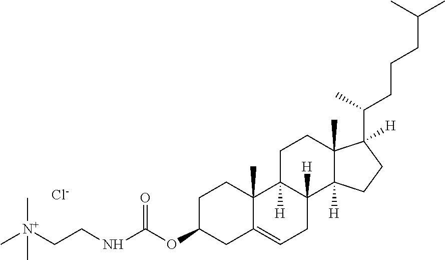

In an embodiment, compound DPhyPE (1,2-(7R,11R) Diphytanoyl-sn-glycero-3-phosphoethanolamine) is of the following formula:

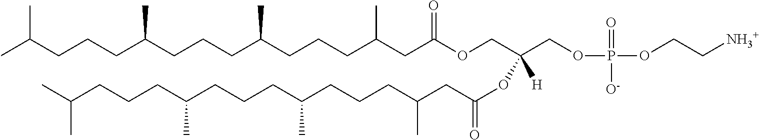 [SMILES: CC(C)CCC[C@@H](C)CCC[C@@H](C)CCCC(C)CC(=O)OC[C@H](COP(=O)([O-])OCC[NH3+])OC(=O)CC(C)CCC[C@H](C)CCC[C@H](C)CCCC(C)C]

In an embodiment, compound DOPE (1,2-Dioleoyl-sn-glycero-3-phosphoethanolamine) is of the following formula:

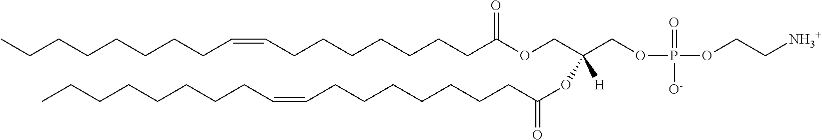 [SMILES: CCCCCCCC/C=C\CCCCCCCC(=O)OC[C@H](COP(=O)([O-])OCC[NH3+])OC(=O)CCCCCCC/C=C\CCCCCCCC]

In an embodiment, compound DLPE (1,2-Dilauroyl-sn-glycero-3-phosphoethanolamine) is of the following formula:

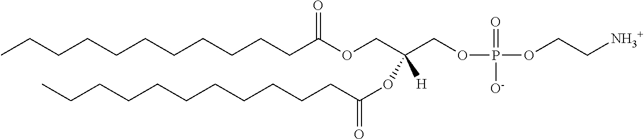 [SMILES: CCCCCCCCCCCC(=O)OC[C@H](COP(=O)([O-])OCC[NH3+])OC(=O)CCCCCCCCCCC]

In an embodiment, compound DMPE (1,2-Dimyristoyl-sn-glycero-3-phosphoethanolamine) is of the following formula:

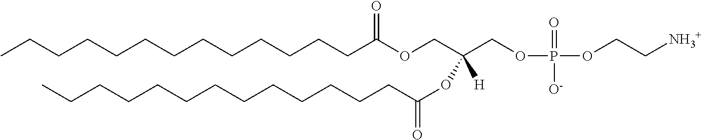 [SMILES: CCCCCCCCCCCCCC(=O)OC[C@H](COP(=O)([O-])OCC[NH3+])OC(=O)CCCCCCCCCCCCC]

In an embodiment, compound POPE ((1-Palmitoyl-2-oleoyl-sn-glycero-3-phosphoethanolamine) is of the following formula:

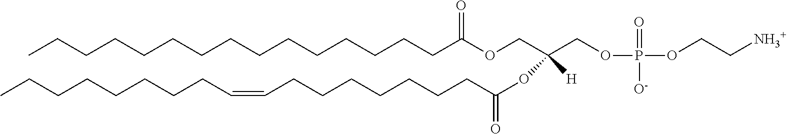 [SMILES: CCCCCCCC/C=C\CCCCCCCC(=O)O[C@H](COC(=O)CCCCCCCCCCCCCCC)COP(=O)([O-])OCC[NH3+]]

In an embodiment, compound DSPC (1,2-Distearoyl-sn-glycero-3-phosphoethanolamine) is of the following formula:

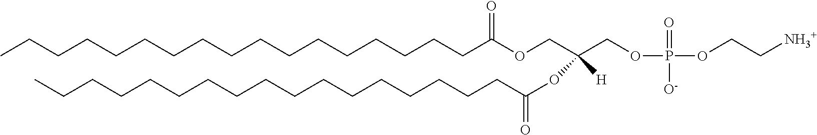 [SMILES: CCCCCCCCCCCCCCCCCC(=O)OC[C@H](COP(=O)([O-])OCC[NH3+])OC(=O)CCCCCCCCCCCCCCCCC]

In an embodiment, compound cholesterol is of the following formula:

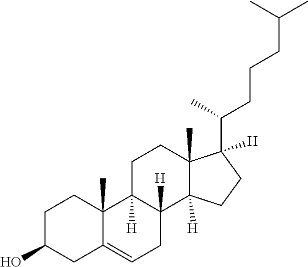

In an embodiment, compound Stigmasterol (Stigmasta-5,22-dien-3-ol) is of the following formula:

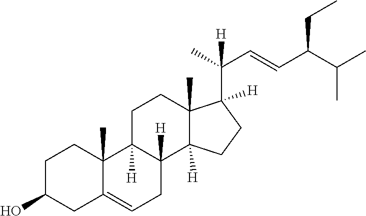

In an embodiment, compound mPEG-500-DSPE (1,2-Distearoyl-sn-glycero-3-phosphoethanolamine-N-[methoxy(polyethylene glycol)-500] (preferably as an ammonium salt) is of the following formula:

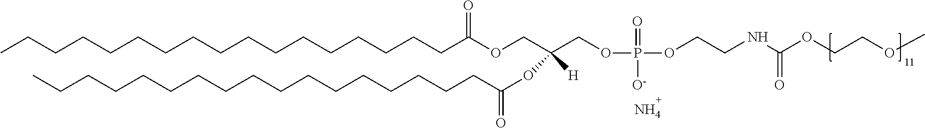

In an embodiment, compound mPEG-1000-DSPE (1,2-Distearoyl-sn-glycero-3-phosphoethanolamine-N-[methoxy(polyethylene glycol)-1000] (preferably as ammonium salt) is of the following formula:

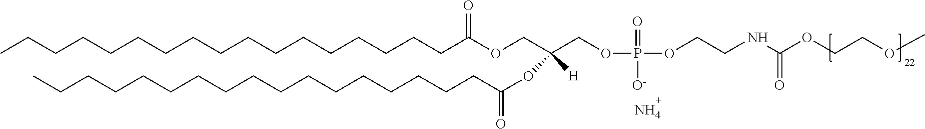

In an embodiment, compound mPEG-2000-DSPE (1,2-Distearoyl-sn-glycero-3-phosphoethanolamine-N-[methoxy(polyethylene glycol)-2000] (preferably as ammonium salt) is of the following formula:

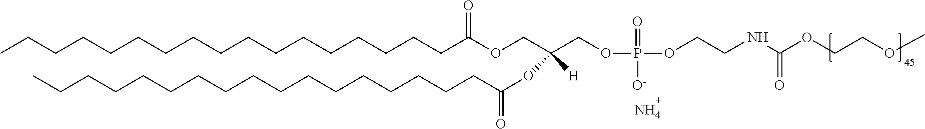

In an embodiment, compound mPEG-5000-DSPE (1,2-Distearoyl-sn-glycero-3-phosphoethanolamine-N-[methoxy(polyethylene glycol)-5000] (preferably as ammonium salt) is of the following formula:

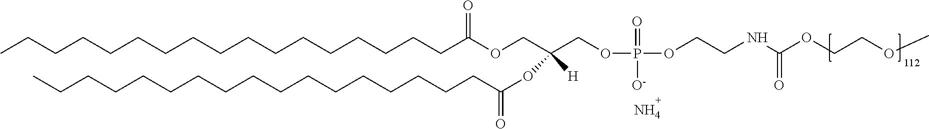

In an embodiment, compound mPEG-DSPE (1,2-Distearoyl-sn-glycero-3-phosphoethanolamine-N-[methoxy(polyethylene glycol)] (preferably as ammonium salt) is of the following formula:

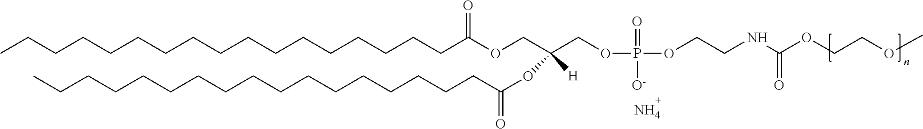

In an embodiment, compound mPEG-2000-DPG (1,2-Dipalmitoyl-rac-glycero-3-methoxypoly ethylene glycol-2000) is of the following formula:

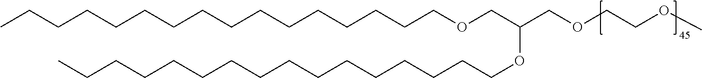

In an embodiment, compound mPEG-2000-DMG (1,2-Dimyristoyl-rac-glycero-3-methoxypolyethylene glycol-2000) is of the following formula:

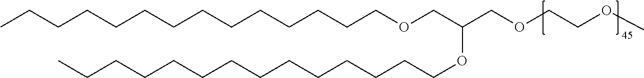

In an embodiment, compound mPEG-2000-DLG (1,2-Dilauroyl-rac-glycero-3-methoxypolyethylene glycol-2000) is of the following formula:

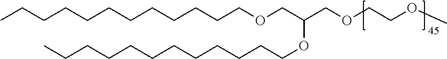

In an embodiment, compound mPEG-2000-C8-Ceramide (N-Octanoyl-sphingosine-1-{succinyl[methoxy(poly ethylene glycol)2000]}) is of the following formula:

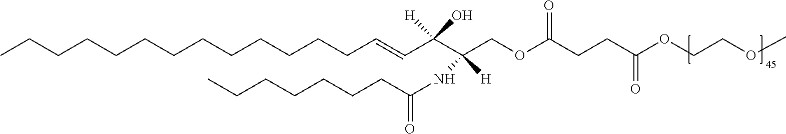

In an embodiment, compound mPEG-2000-C16-Ceramide (N-Palmitoyl-sphingosine-1-{succinyl[methoxy(polyethylene glycol)2000]}) is of the following formula:

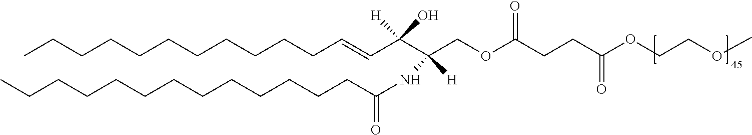

In an embodiment, compound mPEG-2000-C-DMA (N-[(Methoxy poly(ethylene glycol)2000)carbamyl]-1,2-dimyristyloxlpropyl-3-amine) is of the following formula:

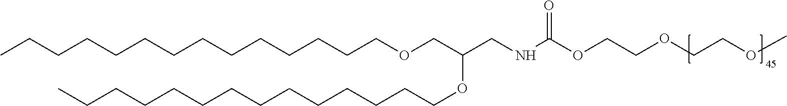

In an embodiment of the first, second and third aspect of the present invention, including any embodiment thereof, and as preferably used herein, a monodisperse composition is a composition where particles formed by a lipid composition of the composition show a monodisperse particle distribution. In an embodiment of the first, second and third aspect of the present invention, including any embodiment thereof, and as preferably used herein, a polydisperse composition is a composition where particles formed by a lipid composition of the composition show a polydisperse particle distribution.

In an embodiment and as preferably used herein, a particle size distribution is monodisperse, if, using a standard dynamic-light-scattering approach, a single peak having a polydispersity index (PDI) from about 0.005 to about 0.17, preferably from about 0.01 to about 0.10 is measured. The respective method is, for example, described in Stetfeld et al. (supra) and Thomas J C et al. (supra). In an embodiment and as preferably used herein, a particle size distribution is polydisperse if, using dynamic-light-scattering, more than one peak and/or a polydispersity index (PDI) of >0.17 is measured. The respective method is, for example, described in Stetfeld et al. (infra).

In an embodiment, particle size of the particles formed by the lipid composition of the composition of the invention is determined by Dynamic Light Scattering using a Zetasizer Ultra (Malvern Panalytical Ltd, Malvern, UK). All measurements are preferable carried out in an aqueous buffer as dispersant, which is a 270 mM sucrose solution buffered with 10 mM TRIS at pH 7.4. Dynamic Light Scattering is, for example, described in more detail in Stetefeld, J., McKenna, S. A. & Patel, T. R. “Dynamic light scattering: a practical guide and applications in biomedical sciences”. Biophys Rev 8, 409-427 (2016); and Thomas, J. C. “The determination of log normal particle size distributions by dynamic light scattering”. Journal of Colloid and Interface Science 117, 187-192 (1987); the disclosure of which is herein incorporated by reference.

More specifically, i.e., in a more preferred embodiment, particle size is determined as “Intensity Mean Peak” which is calculated by the measurement and analysis software ZX Explorer (Malvern Panalytical Ltd, Malvern, UK). The “Intensity Mean Peak” of the composition of the invention comprising an mRNA according to the Dynamic Light Scattering measurements is within a range of (a) from about 30 nm to about 200 nm, preferably from about 40 nm to about 140 nm, and more preferably within a range from about 60 nm to about 120 nm, or (b) from about 30 nm to about 100 nm and preferably from about 30 nm to 60 nm.

In an embodiment, the zeta potential of the particles formed by the lipid composition of the composition of the invention is determined by Laser Doppler Electrophoresis using a Zetasizer Ultra (Malvern Panalytical Ltd, Malvern, UK). All measurements are preferably carried out in an aqueous buffer as dispersant, which is a 270 mM sucrose solution buffered with 10 mM TRIS at pH 7.4. Laser Doppler Electrophoresis is, for example, described in more detail in Clogston, J. D. & Patri, A. K. in “Characterization of Nanoparticles Intended for Drug Delivery” 697, 63-70 (Humana Press, 2011); and Sze, A., Erickson, D., Ren, L. & Li, D. “Zeta-potential measurement using the Smoluchowski equation and the slope of the current—time relationship in electroosmotic flow”, Journal of Colloid and Interface Science 261, 402-410 (2003), the disclosure of which is incorporated herein by reference.

More specifically, i.e. in a more preferred embodiment, zeta potential is determined as “Zeta Mean Peaks”. Both expressions are calculated by the measurement and analysis software ZX Explorer (Malvern Panalytical Ltd, Malvern, UK). The “Zeta Mean Peak” of the composition of the invention comprising an mRNA according to the Laser Doppler Electrophoresis measurements is in a range of about +0 mV to about +80 mV and preferably in a range of about +0 mV to about +45 mV.

In an embodiment and as preferably used herein, mRNA is a polynucleotide preferably comprising from about 200 to about 100.000 nucleotides, preferably from about 750 to about 5000 nucleotides, more preferably comprising about 1000 to 3000 nucleotides. In an embodiment thereof, the mRNA is a single-stranded nucleic acid, whereby some stretches thereof may be part of a or several double-stranded structure(s) within the polynucleotide. Such double-stranded structure is typically formed by two or more stretches of the polynucleotide the nucleotide sequence of which is at least partially complementary to each other. The double-stranded structure is preferable formed or stabilized by Watson-Crick base pairing.

In a further embodiment, the mRNA shows a very basic design in eukaryotic cells and typically comprises, in 5′->3′ direction, a Cap structure, a 5′ untranslated region (5′ UTR), a coding sequence typically starting with a AUG codon attached to a coding sequence (CDS) terminating with a stop codon, a 3′ untranslated region (3′ UTR) and a poly-A-tail.

In a still further embodiment, the mRNA is a single-stranded molecule which may form secondary and/or tertiary structures, where the mRNA folds back on itself

In accordance with the present invention, the mRNA, in an embodiment, is a therapeutically active agent. Useful in the treatment and/or prevention of a disease. In principle, the administered mRNA sequence can cause a cell to make a protein, which in turn could directly treat a disease or could function as a vaccine; more indirectly the protein could interfere with an element of a pathway in such way that the pathway is either inhibited or stimulated, thereby treating or ameliorating a disease.

In an embodiment to the present invention, the mRNA is different from a recombinant nucleic acid construct comprising in 5′ ->3′ direction

a 5′ UTR,

a coding region coding for an effector molecule, and

a 3′ UTR,

wherein the 5′ UTR is selected from the group comprising a 5′ UTR of a gene coding for MCP-1 or a derivative thereof having a nucleotide identity of at least 85%, a 5′ UTR of a gene coding for RPL12s.c. or a derivative thereof having a nucleotide identity of at least 85%, a 5′ UTR of a gene coding for Ang-2 or a derivative thereof having a nucleotide identity of at least 85%, a 5′ UTR of a gene coding for HSP70 or a derivative thereof having a nucleotide identity of at least 85%, a 5′ UTR of a gene coding for H3.3. or a derivative thereof having a nucleotide identity of at least 85%, a 5′ UTR of a gene coding for Galectin-9 or a derivative thereof having a nucleotide identity of at least 85%, a 5′ UTR of a gene coding for GADD34 or a derivative thereof having a nucleotide identity of at least 85%, a 5′ UTR of a gene coding for EDN1 or a derivative thereof having a nucleotide identity of at least 85%, a 5′ UTR of a gene coding for HSP70m5 or a derivative thereof having a nucleotide identity of at least 85%, a 5′ UTR of a gene coding for E-selectin or a derivative thereof having a nucleotide identity of at least 85% a 5′ UTR of a gene coding for ICAM-1 or a derivative thereof having a nucleotide identity of at least 85%, a 5′ UTR of a gene coding for IL-6 or a derivative thereof having a nucleotide identity of at least 85% and a 5′ UTR of a gene coding for vWF or a derivative thereof having a nucleotide identity of at least 85%;
wherein 3′ UTR is selected from the group comprising a 3′ UTR of a gene coding for vWF or a derivative thereof having a nucleotide identity of at least 85%, a 3′ UTR of a gene coding for MCP-1 or a derivative thereof having a nucleotide identity of at least 85% a 3′ UTR of a gene coding for RPL12s.c. or a derivative thereof having a nucleotide identity of at least 85%, a 3′ UTR of a gene coding for HSP70 or a derivative thereof having a nucleotide identity of at least 85%, a 3′ UTR of a gene coding for H3.3. or a derivative thereof having a nucleotide identity of at least 85%, a 3′ UTR of a gene coding for GADD34 or a derivative thereof having a nucleotide identity of at least 85%, a 3′ UTR of a gene coding for EDN1 or a derivative thereof having a nucleotide identity of at least 85%, and a 3′ UTR of a gene coding for IL-6 or a derivative thereof having a nucleotide identity of at least 85%,
wherein the effector molecule is effective in restoring a cellular function of a cell or is effective in exercising a therapeutic effect in or on a cell, and
wherein the recombinant nucleic acid construct is different from a wild type mRNA coding for the effector molecule.

In an embodiment to the present invention, the mRNA is recombinant nucleic acid construct comprising in 5′->3′ direction

a 5′ UTR,

a coding region coding for an effector molecule, and

a 3′ UTR,

wherein the 5′ UTR is selected from the group comprising a 5′ UTR of a gene coding for MCP-1 or a derivative thereof having a nucleotide identity of at least 85%, a 5′ UTR of a gene coding for RPL12s.c. or a derivative thereof having a nucleotide identity of at least 85%, a 5′ UTR of a gene coding for Ang-2 or a derivative thereof having a nucleotide identity of at least 85%, a 5′ UTR of a gene coding for HSP70 or a derivative thereof having a nucleotide identity of at least 85%, a 5′ UTR of a gene coding for H3.3. or a derivative thereof having a nucleotide identity of at least 85%, a 5′ UTR of a gene coding for Galectin-9 or a derivative thereof having a nucleotide identity of at least 85%, a 5′ UTR of a gene coding for GADD34 or a derivative thereof having a nucleotide identity of at least 85%, a 5′ UTR of a gene coding for EDN1 or a derivative thereof having a nucleotide identity of at least 85%, a 5′ UTR of a gene coding for HSP70m5 or a derivative thereof having a nucleotide identity of at least 85%, a 5′ UTR of a gene coding for E-selectin or a derivative thereof having a nucleotide identity of at least 85% a 5′ UTR of a gene coding for ICAM-1 or a derivative thereof having a nucleotide identity of at least 85%, a 5′ UTR of a gene coding for IL-6 or a derivative thereof having a nucleotide identity of at least 85% and a 5′ UTR of a gene coding for vWF or a derivative thereof having a nucleotide identity of at least 85%;
wherein 3′ UTR is selected from the group comprising a 3′ UTR of a gene coding for vWF or a derivative thereof having a nucleotide identity of at least 85%, a 3′ UTR of a gene coding for MCP-1 or a derivative thereof having a nucleotide identity of at least 85% a 3′ UTR of a gene coding for RPL12s.c. or a derivative thereof having a nucleotide identity of at least 85%, a 3′ UTR of a gene coding for HSP70 or a derivative thereof having a nucleotide identity of at least 85%, a 3′ UTR of a gene coding for H3.3. or a derivative thereof having a nucleotide identity of at least 85%, a 3′ UTR of a gene coding for GADD34 or a derivative thereof having a nucleotide identity of at least 85%, a 3′ UTR of a gene coding for EDN1 or a derivative thereof having a nucleotide identity of at least 85%, and a 3′ UTR of a gene coding for IL-6 or a derivative thereof having a nucleotide identity of at least 85%,
wherein the effector molecule is effective in restoring a cellular function of a cell or is effective in exercising a therapeutic effect in or on a cell,
wherein the effector molecule is a mutant effector molecule, wherein the mutant effector molecule is a gain of function mutant effector molecule and/or a hypermorphic mutant effector molecule, and
wherein the recombinant nucleic acid construct is different from a wild type mRNA coding for the effector molecule.

The composition of the invention and particularly the lipid composition of the invention may comprise, in an embodiment, a carrier. Such carrier is preferably a liquid carrier. Preferred liquid carriers are aqueous carriers and non-aqueous carriers. Preferred aqueous carriers are water, an aqueous salt solution, an aqueous buffer system, more preferably the buffer system and/or the aqueous salt solution have a physiological buffer strength and physiological salt concentration(s). Preferred non-aqueous carriers are solvents, preferably organic solvents such as ethanol, tert.-butanol. Without wishing to be bound by any theory, any water miscible organic solvent can, in principle, be used. It is to be acknowledged that the composition, more particularly the lipid composition can thus be present as or form liposomes; when contacted with overall negatively charged compounds, preferably compounds to be delivered by the lipid composition of the invention and mRNA in particular and/or the composition of the invention, the lipid composition of the invention and the composition of the invention form lipoplexes, i.e. a complex that is formed by the electrostatic interaction and the entropic effect based on the release of counter ions and water when a polyanion such as a nucleic acid molecule interact with a cationic lipid or a lipid system that contains beside other lipid components at least one cationic lipid component.

In a further embodiment, the lipid composition of the invention and/or the composition of the invention is present as a lyophilized composition. The thus lyophilized composition allows effective long-term storage of the composition at room temperature.

In accordance with the present invention, a pharmaceutical composition comprises the composition of the present invention. The pharmaceutical composition of the invention comprises a pharmaceutically active compound and optionally a pharmaceutically acceptable carrier. Such pharmaceutically acceptable carrier may, preferably, be selected from the group of carriers as defined herein in connection with the composition according to the present invention. It will be understood by those skilled in the art that any composition as described herein may, in principle, be also used as a pharmaceutical composition provided that its ingredients and any combination thereof is pharmaceutically acceptable. A pharmaceutical composition comprises a pharmaceutically active compound. Such pharmaceutically active compound is, in an embodiment, the mRNA which is encompassed or comprised by the composition of the present invention.

It is within the ordinary skill of a person of the art that the composition, particularly the pharmaceutical composition according to the present invention can be used for various forms of administration, whereby the composition is to be adapted to such forms of administration.

The method of the present invention for preparing the composition of the present invention comprising an mRNA relies on mixing a solution comprising the lipid components of the lipid composition with a solution comprising the mRNA, wherein the mixing is an in-line mixing. The mixing step is performed by the application of a microfluidic mixing device and can particularly either be done by the use of a staggered herringbone mixer device, a Dean Vortex bifurcating mixing device or a microfluidic hydrodynamic mixing device. These devices allow due to their special designed micro channels a rapid, non-turbulent and diffusion based mixing processes. A staggered herringbone mixer and its use in the preparation of particles as preferably contained in the composition of the invention, is, for example, described in Zhigaltsev, I. V. et al. “Bottom-Up Design and Synthesis of Limit Size Lipid Nanoparticle Systems with Aqueous and Triglyceride Cores Using Millisecond Microfluidic Mixing”. Langmuir 28, 3633-3640 (2012), the disclosure of which is incorporated herein by reference. Microfluidic hydrodynamic mixing and its use in the preparation of particles as preferably contained in the composition of the invention, is, for example, described in Krzysztoń, R. et al. “Microfluidic self-assembly of folate-targeted monomolecular siRNA-lipid nanoparticles.” Nanoscale 9, 7442-7453 (2017), the disclosure of which is incorporated herein by reference. Finally, Dean Vortex bifurcating mixing and its use in the preparation of particles as preferably contained in the composition of the invention, is, for example, described in Chen, J. J., Chen, C. H. & Shie, S. R. “Optimal designs of staggered dean vortex micromixers.” Int J Mol Sci 12, 3500-3524 (2011), the disclosure of which is incorporated herein by reference.

The instant invention is further illustrated by the following Examples and Figures from which further features, embodiment and advantages of the invention may be taken, whereby

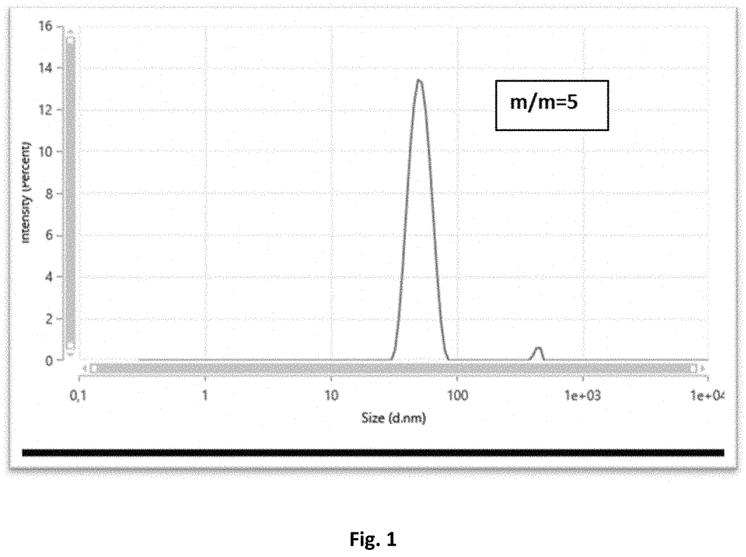

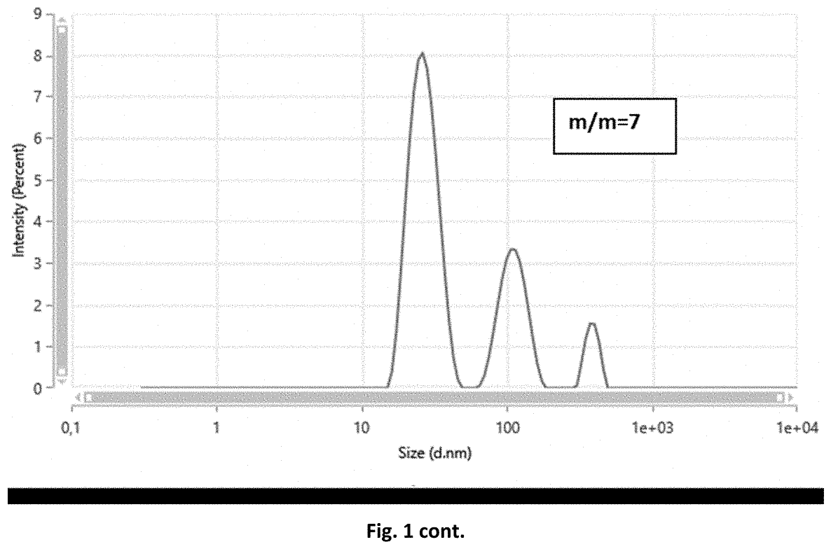

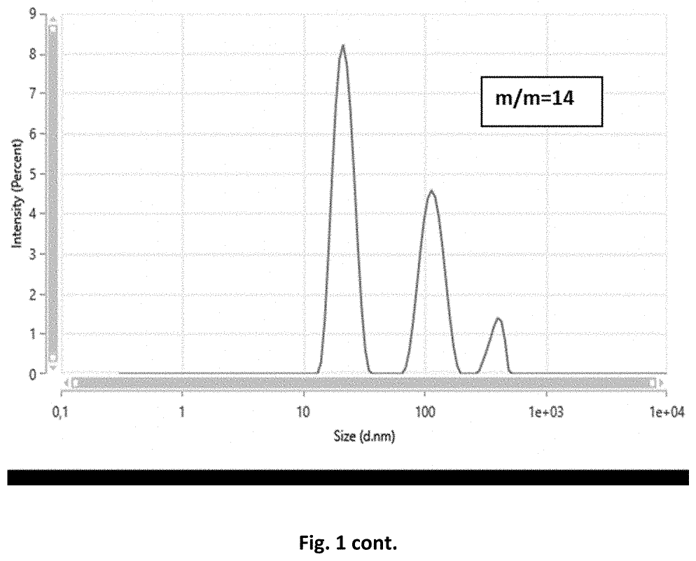

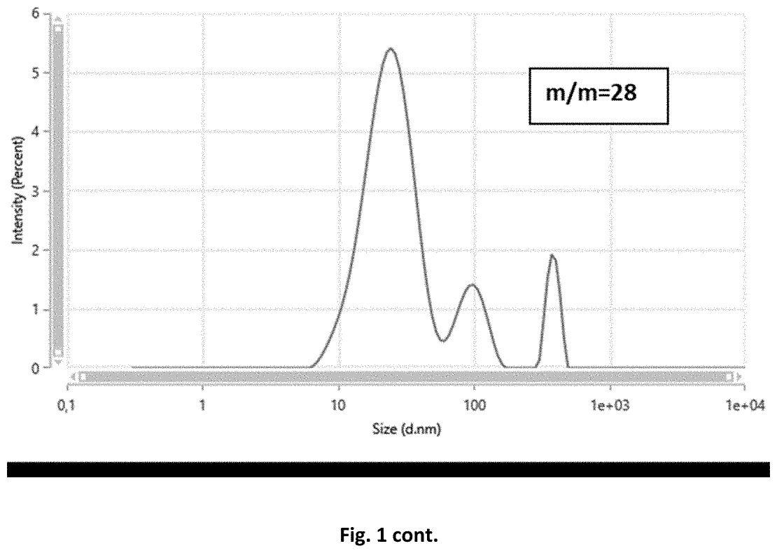

FIG. 1 represents a panel of four diagrams showing at a mass ratio of total lipids to mRNA of 5, 7, 14 and 28 the size distribution of particles of a lipid composition (indicated as “Intensity (Percent)”) having a distinct size (nm) (indicated as “Size (d.nm)”) as determined by multi-angle-dynamic-light-scattering (MADLS);

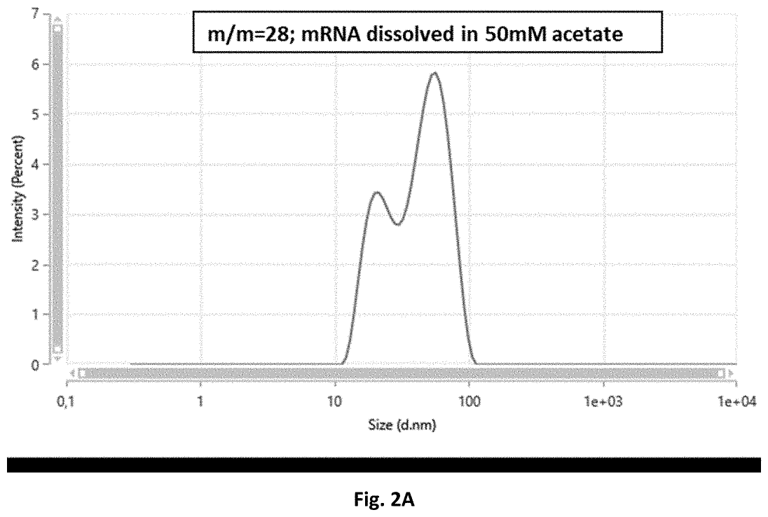

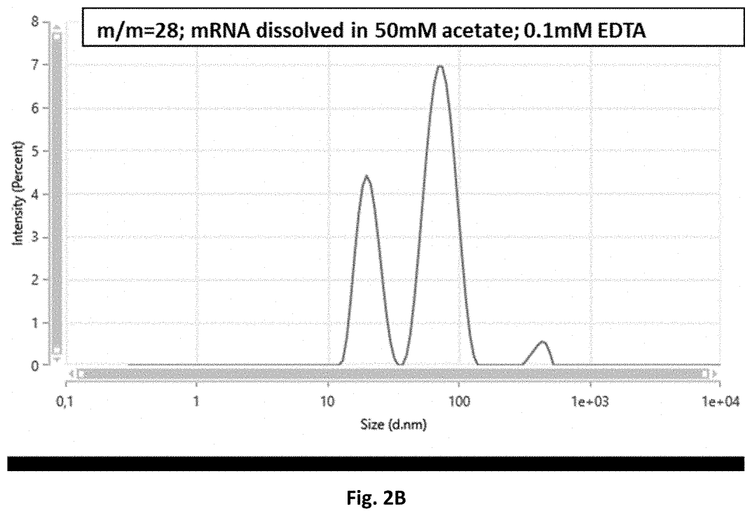

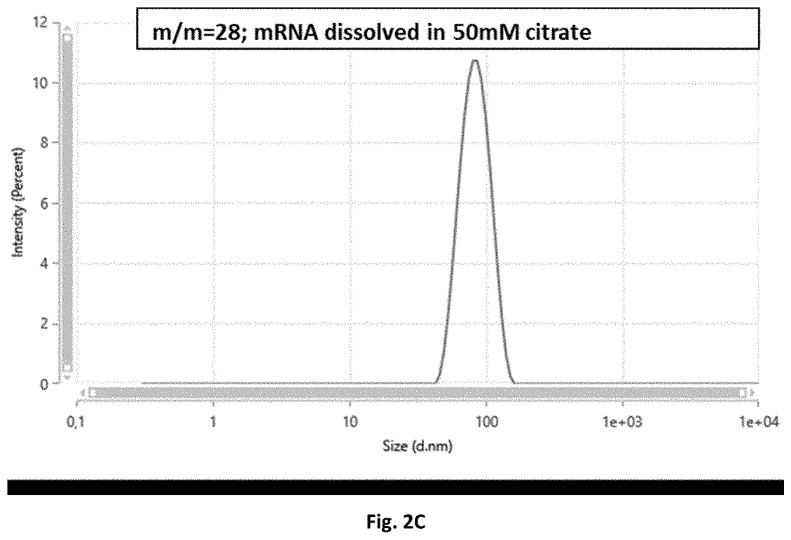

FIGS. 2A, 2B and 2C represent a panel of three diagrams showing the size distribution of particles (indicated as “Intensity (Percent)” having a distinct size (nm) (indicated as “Size (d.nm)”) for various lipid compositions at a mass ratio of total lipids to mRNA of 28, whereby the mRNA was dissolved in a 50 mM acetate buffer (FIG. 2A), in a 50 mM acetate buffer comprising 0.1 mM EDTA (FIG. 2B) or in a 50 mM citrate buffer (FIG. 2C) when mixed with the lipid composition; particle size was determined by multi-angle-dynamic-light-scattering (MADLS);

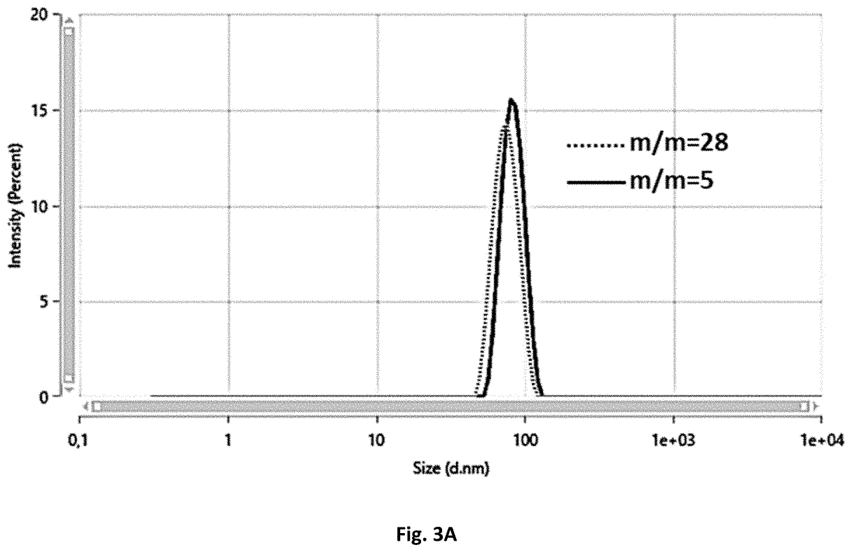

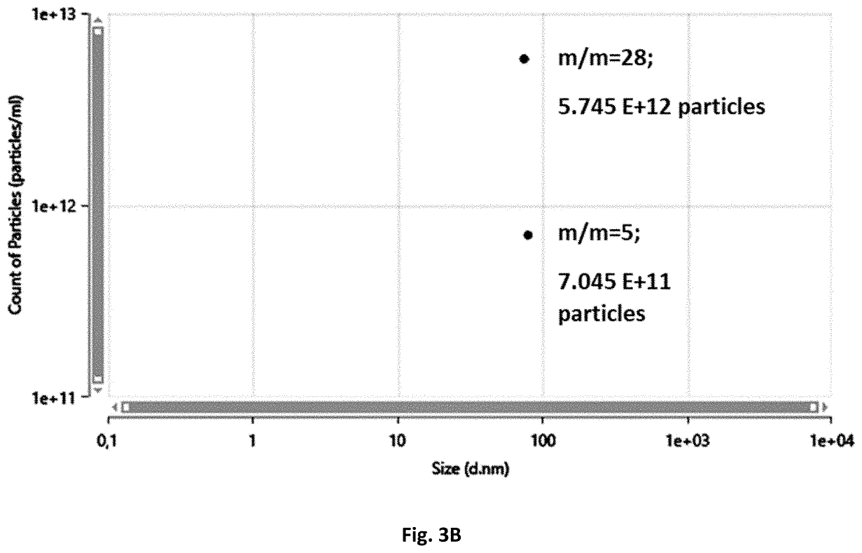

FIG. 3A is a diagram showing the size distribution of particles (indicated as “Intensity (Percent)” having a distinct size (nm) (indicated as “Size (d.nm)”) for two different lipid compositions, wherein a first lipid composition comprises a mass ratio of total lipids to mRNA of 5, and a second mass ratio of total lipids to mRNA of 28; particle size was determined by multi-angle-dynamic-light-scattering (MADLS);

FIG. 3B is a diagram showing particle number of the two different compositions subject to FIG. 3A;

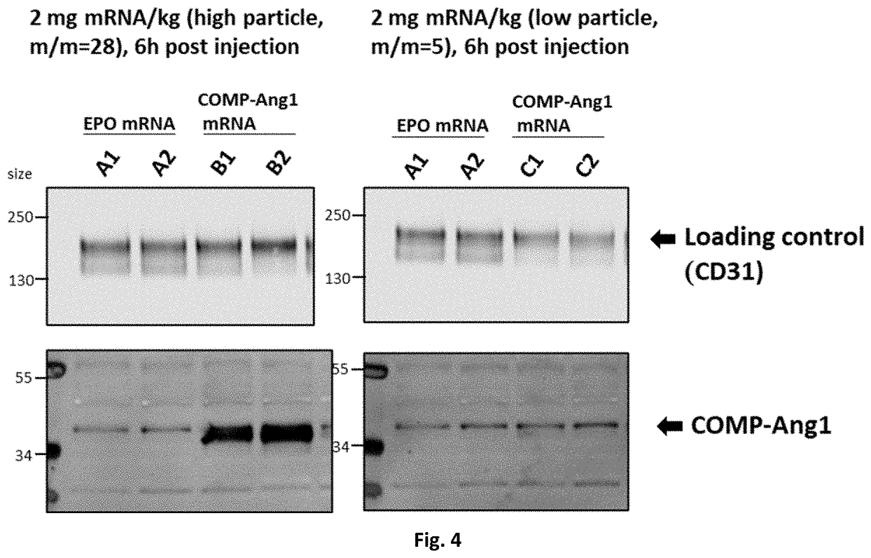

FIG. 4 presents the result of a Western blot analysis for the assessment of COMP-Ang1 protein expression 6 hours post tail vein injection of 2 mg/kg of a composition according to the present invention at a mass ratio of total lipids to mRNA of 28 and 5, respectively, whereby the mRNA content of the composition was 0.2 mg/ml; the mRNA was coding for COMP-Ang1 or—unrelated erythropoietin; CD31 was used as loading control;

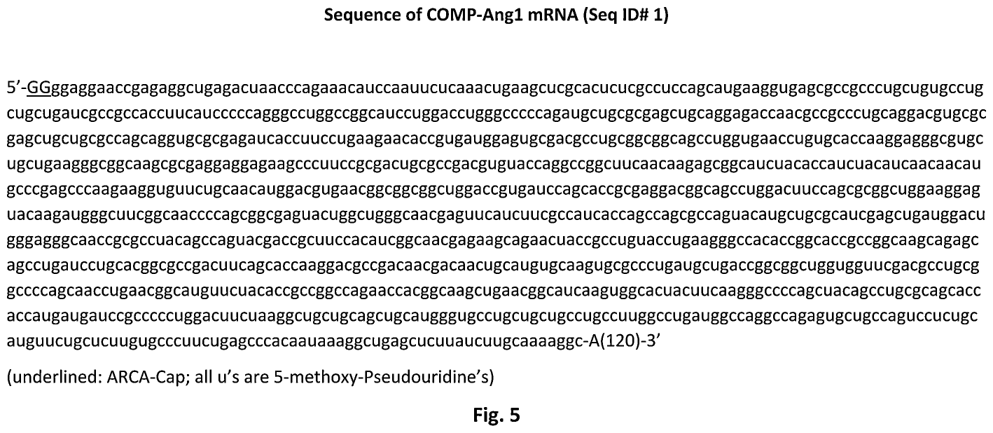

FIG. 5 shows the nucleotide sequence of the mRNA sequence coding for COMP-Ang 1 (SEQ ID NO: 1), wherein the mRNA comprises a 5′-UTR sequence from MCP-1, a signal sequence of MCP-1, the sequence coding for COMP-Ang1 , and the 3′-UTR of von Willebrand factor (vWF), wherein COMP-Ang1 is a designed variant of Angiopoietin-1 (Ang1);

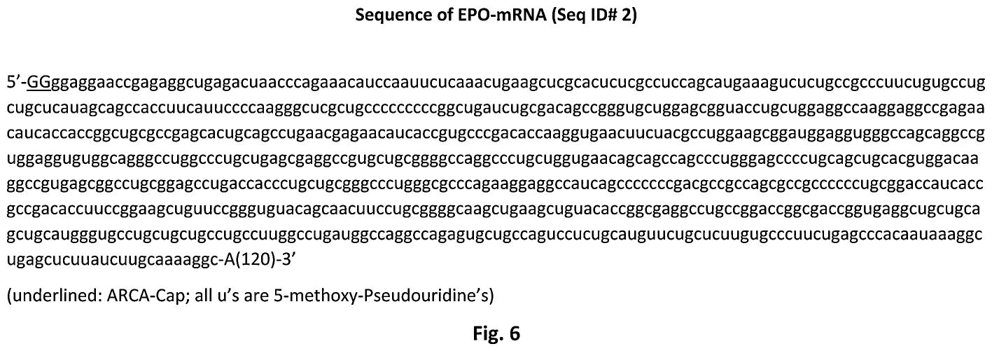

FIG. 6 shows the nucleotide sequence of the mRNA sequence coding for human erythropoietin (EPO) (SEQ ID NO: 2), wherein the mRNA comprises a 5′-UTR sequence from MCP-1, a signal sequence of MCP-1, the sequence coding for EPO, and the 3′-UTR of von Willebrand factor (vW);

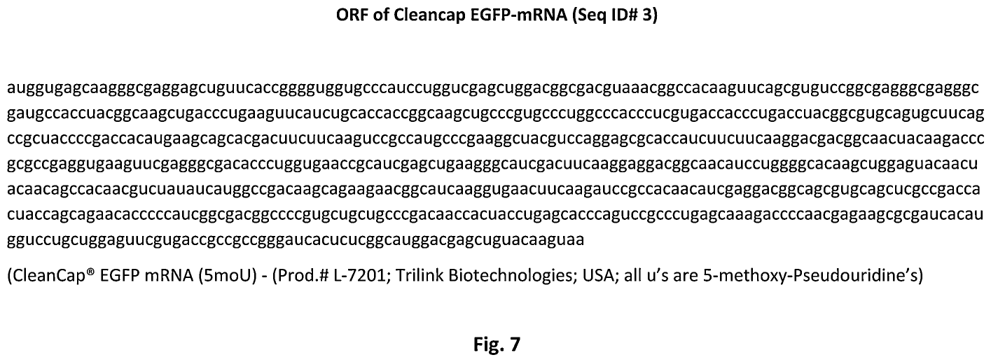

FIG. 7 shows the nucleotide sequence of the open reading frame of Cleancap EGFP-mRNA (as available from Trilink Biotechnologies, USA); and

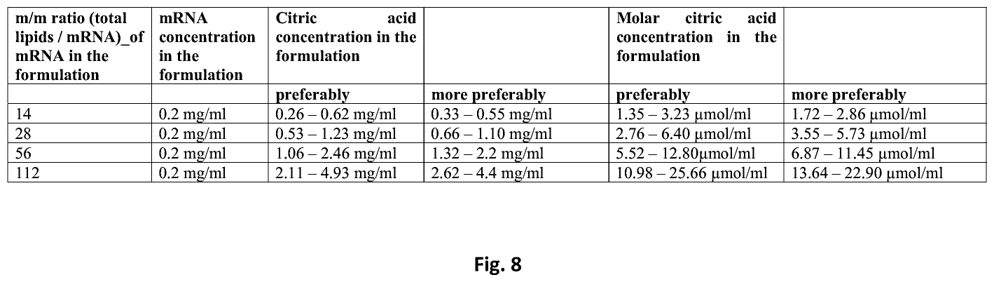

FIG. 8 shows a table indicating various embodiments of the composition of the invention where the mass ratio of total lipid to total nucleic acid molecules, preferably mRNA, in the compositions (“m/m ratio (total lipids/mRNA) of mRNA in the formulation”), concentration of the nucleic acid molecule, preferably mRNA, in the compositions (“mRNA concentration in the formulation”), concentration of citric acid in the compositions in mg/ml (“Citric acid concentration in the formulation”), and molar concentration of citric acid in the compositions (Molar citric acid concentration in the formulation”); in terms of the lipid composition of the compositions having the indicated m/m ratio, mRNA concentration and concentrations of citric acid, such lipid composition is a lipid composition as disclosed herein in connection with the composition according to the first, second and third aspect, including any embodiment thereof, preferably a composition where the molar ratio of the lipids in the lipid composition is
from about 35 mol-% to about 65 mol-% of the cationic lipid,
from about 35 mol-% to about 65 mol-% of the neutral lipid, and
from about 0.1 mol-% to 5 mol-% of the shielding lipid, preferably of the PEGylated lipid,
   wherein the overall lipid content is 100%,
   preferably a composition where the molar ratio of the lipids in the lipid composition is
from about 45 mol-% to about 55 mol-% of the cationic lipid,
from about 45 mol-% to about 55 mol-% of the neutral lipid, and
from about 0.5 mol-% to 2 mol-%,
   wherein the overall lipid content is 100%; and
   more preferably a composition where the molar ratio of the lipids in the lipid composition is
50 mol-% of the cationic lipid, wherein the cationic lipid is (3-(L-Arginyl)-L-2,3-diamino propionic acid-N-palmityl-N-oleyl-amide or L-Arginyl-P-alanine-N-palmityl-N-oleyl-amide,
49 mol-% of the neutral lipid, wherein the neutral lipid is Diphytanoyl-PE (1,2-Diphytanoyl-sn-glycero-3-phosphoethanolamine), and
1 mol-% of the shielding lipid, wherein the shielding lipid is mPEG-2000-DSPE (1,2-distearoyl-sn-glycero-3-phosphoethanolamine-N-[methoxy(polyethylene glycol)-2000] (preferably as sodium salt)).

### EXAMPLE 1

### mRNA Formulation in Cationic Lipid Nanoparticles (LNPs) for In Vivo Applications by Intravenous Administration

For in vivo experiments mRNA-LNPs are prepared in a formulation process with β-(L-Arginyl)-L-2,3-diamino propionic acid-N-palmityl-N-oleyl-amide as cationic lipid. Alternatively, the cationic lipid L-Arginyl-P-alanine-N-palmityl-N-oleyl-amide can be used in an identical procedure to prepare mRNA-LNPs.

Lipids are dissolved in ethanol at appropriate molar ratios (e.g. 50:49:1 β-(L-arginyl)-L-2,3-diamino propionic acid-N-palmityl-N-oleyl-amide: DPyPE: mPEG-2000-DSPE). The lipid mixture is combined with an aqueous solution of mRNA at a volume ratio of 2:1 (aqueous:ethanol) using a microfluidic mixer (NanoAssemblr®; Precision Nanosystems, Vancouver, BC) and flow rates of 18 ml/min. Similarly, LNP formulations can be obtained using citrate or acetate buffered mRNA solutions (pH 3-4). The final total lipid to mRNA mass ratio is 28.

After the mixing process, the formulations are dialyzed against 10 mM HEPES or TRIS buffered isotonic Sucrose solution using 3.5 kDa MWCO Slide-A-Lyzer Dialysis Cassettes (Thermo Fisher Scientific). The formulation in the floating dialysis cassette was dialyzed for 2 hours at room temperature while gently stirring the dialysis buffer; the dialysis buffer was changed and dialyze for another 2 hours at room temperature. Once again, the dialysis buffer was changed and dialyzed at 4° C. overnight. During the course of the dialysis procedure a total of dialysis buffer of at least 300 times the volume of the sample was used. Instead of Sucrose, other sugars like Trehalose or Glucose can be equally used within the formulation process.

Subsequently, the formulations are tested for particle size (Zetasizer Ultra instrument (Malvern Instruments Ltd, Malvern, UK), RNA encapsulation (Quant-iT RiboGreen RNA Assay Kit following manufacturer's (Thermo Fisher Scientific) protocol), and endotoxin and are found to be between 80 to 120 nm in size with a Zeta-potential of >0 mV, display greater than 90% mRNA encapsulation and <1 EU/ml of endotoxin.

The accordingly obtained mRNA-LNP formulations are stored at −80° C. until further in vitro or in vivo use.

### EXAMPLE 2

### Lung-Specific Delivery of mRNA in a Mouse Study

Compositions as prepared in Examples 1 are used in a mouse study as formulations. The mRNA contained in the compositions code for luciferase.

The formulations are administered intravenously through bolus tail-vein injection at a dose of 1 mg mRNA/kg body weight, respectively. Four hours after injection, mice are sacrificed and tissue samples from organs (e.g., liver, lung, spleen, heart, brain, kidney) collected. Luciferase activity in tissue samples is measured by a Luciferase Assay System according to the supplier's protocol (Promega GmbH, Walldorf, Germany).

Luciferase activity was significantly increased in lung tissue compared to spleen tissue, heart tissue, brain tissue, kidney tissue and liver tissue.

### EXAMPLE 3

### Vasculature-Specific Delivery of mRNA in a Cynomolgus Study

A single intravenous infusion of a mRNA (PTX RNA) which is coding for human COMP-Angiopoitin-1 formulated within the lipid system β-(L-Arginyl)-L-2,3-diamino propionic acid-N-palmityl-N-oleyl-amide/DPhyPE/mPEG-2000-DSPE (50 mol %:49 mol %:1 mol %) prepared as described in Example 1, is administered at different doses to cynomolgus monkeys (4 monkeys per dose group, 1 hour infusion). Blood samples are taken at pre-dose and post-dose time points (pre-dose: day 1, day 0, post-dose: t=0.5 h, 1 h, 2 h, 4 h, 8 h, 24 h, 48 h) and aliquoted for pharmacodynamic (PD) and cytokine analysis.

Angiopoietin 1 and Angiopoietin2 levels at those different time points are measured by a standardized and commercially available ELISA assay or by a custom-made ELISA detecting the secreted protein of interest. The corresponding pharmacodynamics parameter are analyzed.

The secreted protein can be measured in a dose and time dependent manner in serum samples of the treated animals. The expression starts at 2 h post infusion and a peak of expression is reached around 6-10 h. The clearance of the protein is dependent on the serum half-life of the secreted protein and/or on the stability of the protein.

This data indicate that the formulation of the invention is a suitable mRNA formulation for protein expression in the vasculature of non-human primates (NHPs) and humans.

### EXAMPLE 4

### mRNA Formulation in Cationic Lipid Nanoparticles (LNPs) for In Vivo Applications by Intravenous Administration m/m Ratio (Total Lipid/mRNA)=28

For in vivo experiments mRNA-LNPs are prepared in a formulation process with β-(L-Arginyl)-L-2,3-diamino propionic acid-N-palmityl-N-oleyl-amide as cationic lipid, Diphytanoyl-PE (1,2-Diphytanoyl-sn-glycero-3-phosphoethanolamine) as co-lipid and methoxyPEG(2000)-DSPE as PEG-lipid.

In a first step, all three lipids are dissolved in ethanol at a molar ratio of 50 mol % β-(L-arginyl)-L-2,3-diamino propionic acid-N-palmityl-N-oleyl-amide, 49 mol % DPyPE and 1 mol % mPEG-2000-DSPE resulting in a total pre-mixing lipid concentration of 18.47 mg/ml. At the same time, the mRNA (COMP-Ang1-mRNA; 1273 nt; SEQ ID NO:1) is dissolved in Citrate buffer (10 mM, pH 5.5) resulting in a pre-mixing mRNA concentration of 0.33 mg/ml.

Subsequently, the pre-mixing lipid solution is combined with the pre-mixing mRNA solution at a volume ratio of 1:2 using a microfluidic mixer (NanoAssemblr®; Precision Nanosystems, Vancouver, BC) at a flow rate of 18 ml/min. Due to the defined pre-mixing concentrations of the lipid and mRNA solutions and the applied volume ratios during the mixing procedure, the resulting mRNA-lipid nanoparticle (LNP) formulation is characterized amongst others by a total lipid to mRNA mass ratio of 28 (m/m ratio (total lipid/mRNA)=28).

Immediately after the mixing process, the formulation is dialyzed against TRIS-buffered isotonic Sucrose solution (10 mM TRIS, 9% Sucrose, pH 7.5) using 3.5 kDa MWCO Slide-A-Lyzer Dialysis Cassettes (Thermo Fisher Scientific) The formulation in the floating dialysis cassette was dialyzed for 2 hours at room temperature while gently stirring the dialysis buffer; the dialysis buffer was changed and dialyze for another 2 hours at room temperature. Once again, the dialysis buffer was changed and dialyzed at 4° C. overnight. During the course of the dialysis procedure a total of dialysis buffer of at least 300 times the volume of the sample was used.

The formulation's total mRNA concentration after the dialysis procedure is quantified by Quant-iT RiboGreen RNA Assay Kit following manufacturer's protocol (Thermo Fisher Scientific) and the appropriate final mRNA concentration of the LNP formulation is adjusted by dilution with TRIS-buffered isotonic Sucrose solution (10 mM TRIS, 9% Sucrose, pH 7.5) (adjusted to 0.2 mg/ml mRNA).

Subsequently, the formulations are tested for particle size (Zetasizer Ultra instrument (Malvern Instruments Ltd, Malvern, UK), RNA encapsulation and total RNA concentration (Quant-iT RiboGreen RNA Assay Kit following manufacturer's (Thermo Fisher Scientific) protocol), and endotoxin and are found to be between 80 to 120 nm in size, display greater than 90% mRNA encapsulation and <1 EU/ml of endotoxin.

The accordingly obtained mRNA-LNP formulation is stored at −80° C. until further in vitro or in vivo use.

### EXAMPLE 5

### mRNA Formulation in Cationic Lipid Nanoparticles (LNPs) for In Vivo Applications by Intravenous Administration m/m Ratio (Lipid/mRNA)=5

For in vivo experiments mRNA-LNPs are prepared in a formulation process with β-(L-Arginyl)-L-2,3-diamino propionic acid-N-palmityl-N-oleyl-amide as cationic lipid, Diphytanoyl-PE (1,2-Diphytanoyl-sn-glycero-3-phosphoethanolamine) as co-lipid and methoxyPEG(2000)-DSPE as PEG-lipid.

In a first step, all three lipids are dissolved in ethanol at a molar ratio of 50 mol % β-(L-arginyl)-L-2,3-diamino propionic acid-N-palmityl-N-oleyl-amide, 49 mol % DPyPE and 1 mol % mPEG-2000-DSPE resulting in a total pre-mixing lipid concentration of 3.32 mg/ml. At the same time, the mRNA (COMP-Ang1 -mRNA; 1273 nt; SEQ ID NO:1) is dissolved in Citrate buffer (10 mM, pH 5.5) resulting in a pre-mixing mRNA concentration of 0.33 mg/ml.

Subsequently, the pre-mixing lipid solution is combined with the pre-mixing mRNA solution at a volume ratio of 1:2 using a microfluidic mixer (NanoAssemblr®; Precision Nanosystems, Vancouver, BC) at a flow rate of 18 ml/min. Due to the defined pre-mixing concentrations of the lipid and mRNA solutions and the applied volume ratios during the mixing procedure, the resulting mRNA-LNP formulation is characterized amongst others by a total lipid to mRNA mass ratio of 5 (m/m ratio (lipid/mRNA)=5).

Immediately after the mixing process, the formulation is dialyzed against TRIS-buffered isotonic Sucrose solution (10 mM TRIS, 9% Sucrose, pH 7.5) using 3.5 kDa MWCO Slide-A-Lyzer Dialysis Cassettes (Thermo Fisher Scientific). The formulation in the floating dialysis cassette was dialyzed for 2 hours at room temperature while gently stirring the dialysis buffer; the dialysis buffer was changed and dialyze for another 2 hours at room temperature. Once again, the dialysis buffer was changed and dialyzed at 4° C. overnight. During the course of the dialysis procedure a total of dialysis buffer of at least 300 times the volume of the sample was used.

The formulation's total mRNA concentration after the dialysis procedure is quantified by Quant-iT RiboGreen RNA Assay Kit following manufacturer's protocol (Thermo Fisher Scientific) and the appropriate final mRNA concentration of the LNP formulation is adjusted by dilution with TRIS-buffered isotonic Sucrose solution (10 mM TRIS, 9% Sucrose, pH 7.5) (adjusted to 0.2 mg/ml mRNA).

Subsequently, the formulations are tested for particle size (Zetasizer Ultra instrument (Malvern Instruments Ltd, Malvern, UK), RNA encapsulation and total RNA concentration (Quant-iT RiboGreen RNA Assay Kit following manufacturer's (Thermo Fisher Scientific) protocol), and endotoxin and are found to be between 70 to 100 nm in size, display greater than 90% mRNA encapsulation and <1 EU/ml of endotoxin.

The accordingly obtained mRNA-LNP formulation is stored at −80° C. until further in vitro or in vivo use.

### EXAMPLE 6

### Quantification of Citric Acid in Cationic Lipid Nanoparticles (LNPs) Using a Citrate-Lyase Based Enzyme Reaction

For quantification of the citric acid content in mRNA-lipid nanoparticles produced as described in Example 4, a citrate-lyase based enzyme kit from Megazyme (K-CITR; Megazyme, Ireland) was used according to the manufacturer's handling instruction. Therefore, 50 μl of the LNP sample (0.2 mg/ml COMP-Ang1-mRNA) prepared in Example 4 was diluted in 950 μl 2% aqueous Triton X 100 to disrupt the nanoparticles. Subsequently, 250 μl buffer (supplied within the kit), 100 μl NADH solution and 1082 l of a mixture of L malate dehydrogenase and D lactate dehydrogenase, supplied within the kit, were added to the diluted sample. After 5min, the optical density of the thus obtained mixture was read at 340 nm against a water blank sample. Thereafter, 10 μl of citrate-lyase supplied in the kit, was added to the mixture and the OD340 nm was read again after 5 min. The absorbance difference of both measurements was used for calculation of the citric acid content in the sample using the following equation:

$$c = \frac{V \times MW}{ε \times d \times v} \times Δ {A}_{citric acid} [ g / L ]$$

where V is the final volume (1.37 ml), MW is the molecular weight of citric acid (192.1 g/mol), ε is the extinction coefficient of NADH at 340 nm (6300 L*mol−1*cm−1), d is the light path (1 cm), v is the sample volume (50 μl) and ΔA the measured absorbance difference (ΔOD(340)=1.065). Using this calculation, a citric acid content of approximately 4.6 μmol/ml (0.88 mg/ml) was measured.

### EXAMPLE 7

### In Vivo Protein Expression of COMP-Ang1 and Erythropoietin After Tail Vein i.v. Injection of Cationic LNPs (m/m=28 and m/m=5) Comprising mRNA Encoding Either for COMP-Ang1 or Erythropoietin

LNPs comprising mRNA either encoding for COMP-Ang1 protein [SEQ ID NO: 1] of human erythropoietin [SEQ ID NO:2] were prepared at a m/m ratio of 28 (high particle concentration) and a m/m ratio of 5 (low particle concentration) (m/m=mass total lipids/mass mRNA) as described in Example 4 and Example 5. LNPs were stored at −80° C. until further use. Subsequently, 8-10 weeks old, male mice (strain C57B1/6) were tail vein injected with 300 μl/mouse (corresponding to 2 mg/kg mRNA) of the prepared LNP-formulation. Animals were sacrificed 6 hours post injection and lung tissue samples of the treated animals were snap-frozen immediately. For Western immunoblot analysis 20-80 mg of the frozen lung tissue samples were homogenized in T-PER Tissue Protein Extraction Reagent (20 μl/mg tissue) (Thermo Scientific, USA) using a bead mill at 50 Hz (TissueLyser L T, Qiagen, Germany).

Lung tissue protein lysates were subsequently separated using SDS-PAGE (4-12% gel) and protein levels were assessed by immunoblot analysis using anti-Ang1 antibody (Recombinant Anti-Angiopoietin 1 antibody [EPR2888(N)] (ab183701), Abcam, Cambridge, UK), and anti-CD31 antibody (CD31 (PECAM-1) (D8V9E) XP® Rabbit mAb #77699; Cell Signaling Technologies, Danvers, Mass., USA) as a loading control. The results are shown in FIG. 4. Six hours after tail vein injection of the LNP-formulation a very strong expression of COMP-Ang1 protein was detectable in the lung tissue samples for the LNPs with an m/m of 28, whereas the LNPs with an m/m of 5 did not show any expression of COMP-Ang1.

### EXAMPLE 8

### Impact of Mass Ratio Total Lipids/mRNA on Particle Size Distribution of a Lipid Composition

For evaluation of the impact of the mass ratio [total lipids/mRNA] mRNA-LNPs were prepared in a formulation process with β-(L-Arginyl)-L-2,3-diamino propionic acid-N-palmityl-N-oleyl-amide as cationic lipid, Diphytanoyl-PE (1,2-Diphytanoyl-sn-glycero-3-phosphoethanolamine) as co-lipid and methoxyPEG(2000)-DSPE as PEG-lipid.

In a first step, all three lipids were dissolved in ethanol at a molar ratio of 50 mol % β-(L-arginyl)-L-2,3-diamino propionic acid-N-palmityl-N-oleyl-amide, 49 mol % DPyPE and 1 mol % mPEG-2000-DSPE in a way that 4 different pre-mixing lipid solutions were resulting having a total lipid concentration of 0.5 mg/ml, 0.7 mg/ml, 1.4 mg/ml and 2.8 mg/ml. At the same time, the mRNA (CleanCap EGFP, Trilink Biotechnologies, San Diego, Calif., USA, SEQ ID NO: 3) was dissolved in water resulting in a pre-mixing mRNA concentration of 0.15 mg/ml.

Subsequently, all 4 the pre-mixing lipid solutions were combined with the pre-mixing mRNA solution at a volume ratio of 1:2 using a microfluidic mixer (NanoAssemblr®; Precision Nanosystems, Vancouver, BC) at a flow rate of 18 ml/min. Due to the defined pre-mixing concentrations of the lipid and mRNA solutions and the applied volume ratios during the mixing procedure, the resulting mRNA-LNP formulations were characterized amongst others by a total lipid to mRNA mass ratio of 5, 7, 14, and 28.

Immediately after the mixing process, the formulations was dialyzed against TRIS-buffered isotonic Sucrose solution (10 mM TRIS, 9% Sucrose, pH 7.5) using 3.5 kDa MWCO Slide-A-Lyzer Dialysis Cassettes (Thermo Fisher Scientific) The formulations in the floating dialysis cassettes were dialyzed for 2 hours at room temperature while gently stirring the dialysis buffer; the dialysis buffer were changed and dialyze for another 2 hours at room temperature. Once again, the dialysis buffer was changed and dialyzed at 4° C. overnight. During the course of the dialysis procedure a total of dialysis buffer of at least 300 times the volume of the sample had to be used. This lipid composition forms particles which are referred to as lipid nanoparticles (LNPs)

Various mass ratios of total lipid to mRNA were realized using this lipid composition and determined by multi-angle-dynamic-light-scattering (MADLS). The mass ratios m/m were 5, 7, 14 and 28.

The results are shown in FIG. 1. As may be taken from FIG. 1, the monodisperse particle distribution at m/m-ratio=5 changed to a polydisperse distribution for larger m/m-ratios of 7, 14, 28 as measured by multi-angle-dynamic-light-scattering (MADLS). The reason for these changes seems to reside in an increasing number of “empty”, heterogeneous lipid aggregates bearing no nucleic acid (mRNA).

In a further experiment, the mass ratio total lipid to mRNA was 28 and the mRNA was dissolved for use in the method for preparing the LNPs in (a) a 50 mM acetate buffer, (b) in a 50 mM acetate buffer containing 0.1 mM EDTA or (c) a 50 mM citrate buffer. Particle size was determined using multi-angle-dynamic-light-scattering (MADLS).

The results are shown in FIGS. 2A, 2B and 2C.

If the mRNA was dissolved in 50 mM citrate buffer (pH=5.5) within the microfluidic mixing step with the organic solution of lipids, the resulting particle size distribution remains strongly monodisperse, even for high m/m ratios total lipids/mRNA of 28. Such an effect cannot be achieved with other organo-acid buffers like for example acetate buffer. In addition, this effect was limited to the use of a multivalent cationic lipid such as β-(L-Arginyl)-L-2,3-diamino propionic acid-N-palmityl-N-oleyl-amide.

A further characterization of the thus prepared LNPs revealed that the citrate was stably entrapped into the LNPs and was not removable during a dialysis step (MWcutoff 3.5 kDa) against 10 mM TRIS/9% sucrose used to remove the organic solvent of the lipids. The amount of entrapped citrate can further be quantified in the final, dialyzed LNP using a citrate-lyase based enzyme kit (K-CITR; Megazyme, Ireland) as described in example 6.

As illustrated in FIG. 3, the present inventors also found that a >10-fold larger, highly monodisperse lipid nanoparticle number was obtained by using a m/m ratio total lipids/mRNA of 28 compared to a m/m ratio total lipids/mRNA of 5.

The features of the present invention disclosed in the specification, the claims and/or the examples may both separately and in any combination thereof be material for realizing the invention in various forms thereof.

1. A composition comprising a lipid composition, a tricarboxylic acid and a nucleic acid molecule, wherein the lipid composition comprises a cationic lipid, a neutral lipid and a shielding lipid, wherein a positive charge excess arising from a larger number of positive charges provided by the cationic lipid molecules in the composition compared to the smaller number of negative charges provided by the nucleic acid molecules in the composition is compensated by the charges provided by the tricarboxylic acid.

2. The composition of claim 1, wherein the composition is a monodisperse composition.

3. The composition of claim 1, wherein the amount of the tricarboxylic acid in the composition is higher than the concentration of the tricarboxylic acid at which the composition is a polydisperse composition.

4. The composition of claim 1, wherein the ratio of the mass of total lipids in the formulation to mass of the nucleic acid molecule, in the composition (m/m ratio ) is from 10 to 140.

5. The composition of claim 4, wherein
(a) the m/m ratio (total lipids/(nucleic acid) is selected from the group consisting of from 10 to 20, from 12 to 16, and 14,
(b) the m/m ratio (total lipids/(nucleic acid ) is selected from the group consisting of from 20 to 40, from 24 to 32, and 28,
(c) the m/m ratio (total lipids/(nucleic acid ) is selected from the group consisting of from 40 to 80, from 48 to 64, and 56, or
(d) the m/m ratio (total lipids/(nucleic acid) is selected from the group consisting of from 80 to 140, from 96 to 128, and 112.

6. The composition of claim 4, wherein the concentration of the tricarboxylic acid in the formulation is
(a) from 1.35 μmol/ml to 3.23 μmol/ml, or from 1.72 μmol/ml to 2.86 μmol/ml;
(b) from 2.76 μmol/ml to 6.40 μmol/ml, or from 3.55 μmol/ml to 5.73 μmol/ml;
(c) from 5.52 μmol/ml to 12.80 μmol/ml, or from 6.87 μmol/ml to 11.45 μmol/ml; or
(d) from 10.98 μmol/ml to 25.66 μmol/ml, or from 13.64 μmol/ml to 22.90 μmol/ml.

7. The composition of claim 1, wherein the composition comprises an amount of a nucleic acid molecule, wherein the amount of the nucleic acid molecule, of the composition is selected from the group consisting of from about 0.01 mg/ml to about 1.5 mg/ml, from about 0.05 mg/ml to 0.8 mg/ml, from about 0.075 mg/ml to about 0.4 mg/ml, and about 0.2 mg/ml.

8. The composition of claim 1, wherein the lipid composition forms particles.

9. The composition of claim 8, wherein the particles comprise the tricarboxylic acid, and wherein optionally the tricarboxylic acid forms a complex with the lipid composition, or the tricarboxylic acid is bound by, bound in or bound to the particles.

10. The composition of any claim 1, wherein the particle size is selected from the group consisting of from 30 nm to 200 nm, from about 40 nm to about 140 nm, and from about 60 nm to 120 nm.

11. The composition of claim 10, wherein the tricarboxylic acid is selected from the group comprising citric acid, isocitric acid (1-hydroxypropane-1,2,3-tricarboxylic acid), cis-aconitic acid, trans-aconitic acid, a mixture of both cis-aconitic acid and trans-aconitic acid, propane-1,2,3-tricarboxylic acid (tricarballylic acid), agaric acid, trimesic acid, and any mixture thereof.

12. The composition of claim 1, wherein the molar ratio of the lipids in the lipid composition is
from about 20 mol-% to about 80 mol-% of the cationic lipid,
from about 10 mol-% to about 70 mol-% of the neutral lipid, and
from about 0.1 mol-% to about 10 mol-% or from about 1 mol-% to about 10 mol-% of the PEGylated lipid,
wherein the overall lipid content is 100%.

13. The composition of claim 1, wherein the molar ratio of the lipid composition is
50 mol-% of the cationic lipid, wherein the cationic lipid is β-(L-Arginyl)-L-2,3-diamino propionic acid-N-palmityl-N-oleyl-amide or L-Arginyl-P-alanine-N-palmityl-N-oleyl-amide,
49 mol-% of the neutral lipid, wherein the neutral lipid is Diphytanoyl-PE (1,2-Diphytanoyl-sn-glycero-3-phosphoethanolamine), and
1 mol-% of the shielding lipid, wherein the shielding lipid is mPEG-2000-DSPE (1,2-distearoyl-sn-glycero-3-phosphoethanolamine-N-[methoxy(polyethylene glycol)-2000] (preferably as sodium salt)).

14. The composition of claim 1, wherein the nucleic acid molecule is an mRNA molecule.

15. A method for the treatment and/or prevention of a disease selected from the group comprising acute respiratory distress syndrome, acute lung injury, lung cancer, pulmonary metastasis, pulmonary hypertension and pulmonary artery hypertension, comprising administering to a subject suffering from said disease an effective amount of a composition according to claim 1.

16. A pharmaceutical composition comprising a composition according to claim 1 and a pharmaceutically active agent.

17. A method for preparing a composition according to claim 1, wherein the method comprises mixing a solution comprising the lipid components of the lipid composition with a solution comprising the nucleic acid molecule, preferably the mRNA molecule, in a buffer of a tricarboxylic acid, wherein preferably the mixing is an in-line mixing.

18. The method of claim 17, wherein the mixing is a non-turbulent and diffusion-based mixing, and optionally is a rapid non-turbulent and diffusion-based mixing.

19. The method of claim 17, wherein the reaction mixture obtained upon the mixing is subjected to (a) a dialysis step, (b) a diafiltration step and/or (c) a tangential flow filtration step, wherein the dialysis step, the diafiltration step and/or the tangential flow filtration step are optionally followed by a subsequent concentration step, providing a final reaction mixture, wherein the final reaction mixture is a ready-to-use composition.
